(19) United States
(12) Patent Application Publication
(10) Pub. No.: US 2006/0121063 A1
(43) Pub. Date: Jun. 8, 2006

(54) Group 2 mite polypeptide variants

(75) Inventors: Erwin Ludo Rogeen, Lyngby (DK); Esben Peter Friis, Valby (DK); Nanna Kristensen Soni, Kobenhavn O (DK); Henriette Draborg, Allerod (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 11/256,589

(22) Filed: Oct. 21, 2005

Related U.S. Application Data
(60) Provisional application No. 60/621,742, filed on Oct. 25, 2004.

(30) Foreign Application Priority Data
Oct. 22, 2004 (DK) PA 2004 01620

(51) Int. Cl. A61K 39/35; C07H 21/04; C12N 5/06; C07K 14/435

(52) U.S. Cl. 424/275.1; 530/350; 435/69.3; 435/348; 435/320.1; 536/23.5

(57) ABSTRACT

This invention concerns variants of group 2 mite polypeptides, wherein the polypeptide of the variant comprise one or more mutations in the positions or corresponding to the positions consisting of D64, V40, E53, S57, K82, G83, I97 of SEQ ID NO: 1 or 64, 40, 53, 57, 82, 83, 97 of the Der p 2 polypeptide or the positions G32, D59, L61, E62, A98 of SEQ ID NO: 2 or 32, 59, 61, 62, 98 of the Der f 2 polypeptide.

### CROSS-REFERENCE TO RELATED APPLICATIONS

This application claims priorty from Danish application no. PA 2004 01620 filed Oct. 22, 2004 is claimed under 35 U.S.C. 119(a)-(d) and the benefit of U.S. provisional application No. 60/621,742 filed Oct. 25, 2004 is claimed under 35 U.S.C. 119(e).

### REFERENCE TO SEQUENCE LISTING

The present application contains information in the form of a sequence listing, which is appended to the application and also submitted on a data carrier accompanying this application. The contents of the data carrier are fully incorporated herein by reference.

### FIELD OF THE INVENTION

The present invention relates to variants of the group 2 mite polypeptide antigens allergens having an altered antigenic profile, compared to the parent group 2 polypeptide allergens, processes for making such variants, compositions comprising the variants and use of the variants in immuno-therapy such as allergy vaccination and/or desensitisation.

### BACKGROUND OF THE INVENTION

1. Field of the Invention

The present invention relates to variants of group 2 mite polypeptide antigens, including Der p 2 and Der f 2, comprising a mutation in a minimal epitope and thus having an altered immunogenic profile in exposed animals, including humans.

2. Description of Related Art

Antigenic polypeptides heterologeous to humans and animals, such as the group 2 mite polypeptide allergens, present e.g., in excrements of dust mites Dermatophagoides pteronyssinus (Der p 2) or Dermatophagoides farinae (Der f 2), can induce immunological responses in susceptible individuals, such as an atopic allergic response, in humans and animals. Allergic responses may range from hay fever, rhinoconjunctivitis, rhinitis, and asthma, and in cases when the sensitised individual is exposed, e.g., to bee sting or insect bites, even to systemic anaphylaxis and death.

An individual may become sensitised to such polypeptides, termed allergens, by inhalation, direct contact with skin or eyes, ingestion or injection. The general mechanism behind an allergic response is divided into a sensitisation phase and a symptomatic phase. The sensitisation phase involves a first exposure of an individual to an allergen. This event activates specific T- and B-lymphocytes, and leads to the production of allergen specific antibodies, such as immunoglobulin E (IgE). The specific IgE antibodies bind to IgE receptors on mast cells and basophils, among others, and the symptomatic phase is initiated upon a second exposure to the same or a homologous allergen. The allergen will bind to the cell-bound IgE, and the polyclonal nature of the antibodies results in bridging and clustering of the IgE receptors, and subsequently in the activation of mast cells and basophils. This activation results in the release of various chemical mediators, such as histamine, heparin, proteases, prostaglandin D2 and leukotrienes, involved in the early as well as late phase reactions of the symptomatic phase of allergy.

For certain forms of IgE-mediated allergies, a therapy exists, called specific allergy vaccination (SAV) or immuno therapy (IT), which comprises repeated parenteral or mucosal (e.g., sublingual) administration of allergen preparations formulated as a vaccine (Int. Arch. Allergy Immunol., 1999, vol. 119, pp 1-5). This leads to reduction of the allergic symptoms, most likely due to induction of a protective, non IgE-based immune response, possibly by modulation of the existing Th2 response and/or a redirection of the immune response towards the immunoprotective (Th1) pathway (Int. Arch. Allergy Immunol., 1999, vol. 119, pp 1-5).

Compared to other types of vaccination, allergy vaccination is complicated by the presence of an existing and ongoing immune response in the allergic patients. The presence of allergen specific IgE antibodies on effector cells, such as mast cells and basophils, in affected tissues, may result in allergic symptoms upon exposure to antigens. Thus, the inherent risk of adverse events or side effects limits the antigen dose, which can be administered, and has necessitated prolonged (12-36 months) and cumbersome treatment regimes where the delivered dose slowly is increased over time.

There is thus a need to provide modified allergens, with a lower inherent risk of inducing adverse events, which can be used for specific allergy vaccination. These modified allergens should have a reduced capacity for binding, and especially cross-linking, antigen-specific IgE molecules. At the same time it is important that they retain the tertiary structure, and to some degree the immunogenicity, of the parent allergen, in order to be able to elicit the protective (IgG-based) immune response in the patient.

In order to produce such modified proteins it is desirable to first identify the minimal B cell epitopes on the molecule. An epitope is the structural area on a complex antigen that can combine with an antibody, while the minimal epitope contains the amino acids involved directly in antibody binding.

B-cell epitopes can in nature be continuous, discontinuous or a combination thereof, but must contain around 10 amino acids in order to elicit an antibody response. One may identify larger regions or areas of the molecule comprising an epitope and a minimal epitope, but when desiring to alter immunogenic properties of a polypeptide by introducing mutations in the molecule one will realize the importance of firstly identifying the minimal epitope because it is far less feasible to prepare modified polypeptides by mutating amino acids if the number of amino acids, which potentially is to be mutated, exceeds 5-10 amino acids. This is because the number of possible variants increases steeply with the number of amino acids involved in the mutation strategy (many more permutations possible) and because it may be less useful to modify amino acids which are not part of an epitope.

Several studies have been aimed at identifying epitopes in group 2 dust mite allergens:
Thomas, W R, Smith W A, Hales, B J, Mills, K, O'Brien, R M (2002) Intl Arch Allergy Immunol 129: 1-18;
Chua, K Y, Greene W K, Kehal, P, Thomas, W R (1991) Clin Exp Allergy 21: 161-166;
Van't Hof, W, van den Berg M, Driedijck, P C, Aalberse, R (1993) Int Arch Appl Immunol 101: 437-441;
Smith A M, Chapman M D (1997) Clin Exp Allergy 27: 593-599; and
Van't Hof, W (1991) Mol Immunol 28:1225-1232.

The present invention provides, by application of a tool for in silico identification of epitope patterns, the identification of positions of amino acids hitherto not identified as contributing to the epitopes of group 2 mite allergens, and thus elucidates more complet epitopes suitable for mutation with the purpose of reducing the antigenicity of these polypeptides.

### SUMMARY OF THE INVENTION

Attempts to reduce the allergenicity of polypeptide allergens have been conducted. It was found that small changes in the epitope, may affect the binding to an antibody. This may change the properties of such an epitope, e.g., by converting it from a high affinity to a low affinity epitope towards antibodies, or even result in epitope loss, i.e. that the epitope cannot sufficiently bind an antibody to elicit an antigenic response.

In order to produce modified group 2 mite polypeptides with improved properties as a vaccine agent, it is an advantage to first identify the minimal B cell epitopes on the molecule. An epitope is the smallest structural area on a complex antigen that can bind an antibody. B-cell epitopes can be continuous or discontinuous in nature. The minimal epitope consists of the specific amino acids directly involved in antibody binding.

The present invention relates to variants of group 2 mite polypeptide antigens, including Der p 2 and Der f 2, comprising a mutation in a minimal epitope and thus having an altered immunogenic profile in exposed animals, including humans. In a particular embodiment the parent group 2 mite polypeptide is a native group 2 mite polypeptide.

In a first aspect the present invention relates to a variant of a parent group 2 mite polypeptide, wherein the polypeptide of the variant comprise one or more mutations in the parent polypeptide in the positions or corresponding to the positions consisting of D64, V40, E53, S57, K82, G83, I97 of SEQ ID NO: 1 or 64, 40, 53, 57, 82, 83, 97 of the Der p 2 polypeptide or the positions G32, D59, L61, E62, A98 of SEQ ID NO: 2 or 32, 59, 61, 62, 98 of the Der f 2 polypeptide.

In a further aspect the present invention relates to variants having an altered IgE-antigenicity as compared to the parent group 2 mite polypeptide, said variants comprising one or more of the mutations D64, V40, S57, I97 of SEQ ID NO: 1.

In a still further aspect the present invention relates to variants having an altered IgG-antigenicity as compared to the parent group 2 mite polypeptide, said variants comprising one or more of the mutations D64, E53, K82, G83 of SEQ ID NO: 1.

In a particular embodiment the variant comprises the mutation D64 of SEQ ID NO: 1, or the mutation L61 of SEQ ID NO: 2.

Further the present invention relates to variants of group 2 mite polypeptides having at least the same T-cell stimulatory effect compared to the parent group 2 mite polypeptide.

In a further embodiment the parent group 2 mite polypeptide has in its mature form a sequence which displays at least 80% identity to SEQ ID NO:1, preferably at least 85% identity, in particular at least 90% identity, such as at least 95% identity, more particularly 98% identity, more particularly 100% identity to SEQ ID NO:1 or 100% identity to Der p 2.

In a further embodiment the parent group 2 mite polypeptide has in its mature form a sequence which displays at least 80% identity to SEQ ID NO:2, preferably at least 85% identity, in particular at least 90% identity, such as at least 95% identity, more particularly 98% identity, more particularly 100% identity to SEQ ID NO:2 or 100% identity to Der f 2.

In a preferred embodiment, the variant of the parent Der p 2 polypeptide has an amino acid sequence which is at least 70% identical, more preferably at least 75% identical, even more preferably at least 80% identical, in particular at least 85% identical, such as at least 90% identical, and most preferably at least 95% identical to the amino acid sequence of the parent.

In a preferred embodiment, the variant of the parent Der p 2 polypeptide of SEQ ID NO: 1 has an amino acid sequence which is at least 70% identical, more preferably at least 75% identical, even more preferably at least 80% identical, in particular at least 85% identical, such as at least 90% identical, and most preferably at least 95% identical to the amino acid sequence of SEQ ID NO: 1.

In a preferred embodiment, the variant of the parent Der f 2 polypeptide has an amino acid sequence which is at least 70% identical, more preferably at least 75% identical, even more preferably at least 80% identical, in particular at least 85% identical, such as at least 90% identical, and most preferably at least 95% identical to the amino acid sequence of the parent.

In a preferred embodiment, the variant of the parent Der f 2 polypeptide of SEQ ID NO: 2 has an amino acid sequence which is at least 70% identical, more preferably at least 75% identical, even more preferably at least 80% identical, in particular at least 85% identical, such as at least 90% identical, and most preferably at least 95% identical to the amino acid sequence of SEQ ID NO: 2.

The present invention further relates to a variant, wherein the mutation of the parent group 2 mite polypeptide comprises substitution of a hydrophilic amino acid to a hydrophobic amino acid, a polar amino acid to a non-polar amino acid, or an acidic amino acid to a basic amino acid.

Further the present invention relates to variants wherein mutation in the parent group 2 mite polypeptide comprise insertion of one or more attachment groups for conjugating a polymer or of one or more glycosylation sites.

In further aspects the invention provides a nucleotide sequence encoding the variant of the invention; a nucleotide construct comprising the nucleotide sequence encoding the variant, operably linked to one or more control sequences that direct the production of the variant in a host cell; a recombinant expression vector comprising the nucleotide construct of the invention and to a recombinant host cell comprising the nucleotide construct of the invention.

In a still further aspect the invention provides a method of preparing a variant of the invention comprising:

(a) cultivating a recombinant host cell of the invention under conditions conducive for production of the variant of the invention and

(b) recovering the variant.

In still further aspects the invention provides a composition comprising a variant of the invention and a pharmaceutically acceptable carrier and a method for preparing such a pharmaceutical composition comprising admixing the variant of the invention with an acceptable pharmaceutical carrier.

In still further aspect the invention provides a variant or a composition of the invention for use as a medicament.

In still further aspect the invention provides use of a variant or the composition of the invention for the preparation of a medicament for the treatment of an immunological disorder.

### DETAILED DESCRIPTION OF THE INVENTION

Definitions

The term “native polypeptide” as used herein is to be understood as a polypeptide essentially in its naturally occurring form. A native polypeptide may be for example be a wild type polypeptide, i.e. a polypeptide isolated from the natural source, a polypeptide in its naturally occurring form obtained via genetic engineering by expression in host organism different from the natural source or by polypeptide synthesis.

An “epitope” or a “B-cell epitope”, as used in this context, is an antigenic determinant and the structural area on a complex antigen that can combine with or bind an antibody. It can be discontinuous in nature, but will in general have a size of 1 kD or less (about 10 amino acids or less). The size may be 3 to 10 amino acids or 5 to 10 amino acids or even 7 to 10 amino acids, depending on the epitope and the polypeptide.

The term “epitope pattern” as used herein is to be understood as a consensus sequence of antibody binding peptides. An example is the epitope pattern A R R * R. The sign “*” in this notation indicates that the aligned antibody binding peptides included a non-consensus moiety between the second and the third arginine. That moiety may be any amino acid or a few amino acids or no amino acid. Epitope patterns are used to identify epitopes and minimal epitopes on complex antigens.

The term “Anchor amino acid” as used herein is to be understood as conserved individual amino acids of an epitope pattern recurring in all peptides bound by monospecific antibodies used to define that pattern. Anchor amino acid will usually also be the amino acid of a minimal epitope on the full polypeptide.

The “antigenicity” of a polypeptide indicates, in this context, its ability to bind antibodies e.g., of IgE and/or IgG and/or other immunoglobulin classes. The ‘IgE-antigenicity’ of a polypeptide as used herein, indicates its ability to bind IgE antibodies.

The “immunogenicity” of a polypeptide indicates its ability to stimulate antibody production and immunological reactions in exposed animals, including humans.

The “allergenicity” of a polypeptide indicates its ability to stimulate IgE antibody production and allergic sensitization in exposed animals, including humans.

The term “parent” or “parent group 2 mite polypeptide” is to be understood as a group 2 mite polypeptide before introducing the mutations according to the invention. In particular the parent group 2 mite polypeptide is the native group 2 mite polypeptide.

For purposes of the present invention, the degree of identity, that is the “% identity” between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10 and gap length penalty of 10. Pairwise alignment parameters were Ktuple=1, gap penalty=3, windows=5, and diagonals=5.

Group 2 Mite Polypeptides

Group 2 allergens were described from house mites Dermatophagoides pteronyssinus (Der p) and Dermatophagoides farinae (Der f), and from storage mites Lepidoglyphus destructor (Lep d), Glycophagus domesticus (Gly d) and Tyrophagus putrescentiae (Tyr p).

The group 2 allergens were first characterized as 14,000-18,000 MW allergens with a high IgE-binding activity. The cDNA sequences of Der p 2 and Der f 2 showed that the allergens had 129 residues, a calculated MW of 14,000 and no N-glycosylation sites. Der p 2 and Der f 2 had 12% amino acid divergence which was evenly distributed throughout the sequence. The biochemical function of the group 2 allergens is still unknown. There was some speculation that Der p 2 may be a lysosyme, but the tertiary structure has made it clear that it is not. Recently the group 2 allergens have been shown to have similarity in sequence, size and distribution of cysteine residues with a family of epididymal proteins.

The identity among group 2 allergens is significantly higher than the group I allergens, and substitutions are more conservative. Only one third of the substitutions were non-conservative compared with the opposite ratio for the group I allergens. The Eur m 2 has 82% sequence identity to both preferential similarity to either allergen.

80% of sera from mite-sensitive patients reacted with Der p 1 and 2 and that these allergens inhibited almost all the IgE anti-HDM reactivity of a pool of positive sera. Der p 2 and Der f 2 have been reported to have almost complete cross-reactivity, although some difference has been noted. The cross-reactivity of Der p 2, Der f 2 and Eur m 2 can be seen by the conservation of surface residues. The minimal cross-reactivity between Der p 2 and Lep d 2, Tyr p 2 and Gly d 2 seem to relate to multiple substitutions across the surface.

Studies with Der p 2 revealed that the pattern of polymorphism was distinct from the sporadic changes described for Der p 1. The described variants of Der p 2 showed a clear pattern of divergence. The most abundant sequence was the variant Der p 2.0101 which was the first sequence described for Der p 2. About half the clones had this sequence. Most changes to the sequence involved a pattern of substitution where residues 40, 47, 111 and 114 were substituted from VTMD in Der p 2.0101 to LSLN in Der p 2.0104. Natural Der p 2 would be best represented by a mixture of the sequence Der p 2.0101 and Der p 2.0104 which contains all of the common substitutions.

The regions of Der p 2 most frequently recognized by T-cell responses were within residues 61-86 and 78-104 and would not be affected by the substitutions. A major region of recognition was however found with peptide 111-129 which induced proliferation in cells from 16/24 subjects and this may be affected by the close changes at positions 111 and 114. A study of proliferative responses induced with recombinant variants found that they all were all stimulatory but some differences could be detected especially when cytokine production was measured.

An important result for the study however was that a mixture of the main variants Der p 2.0101 and Der p 2.0104 would provide a good representation of responses to natural Der p 2.

In Silico Identification of Epitope Patterns and Epitopes in Group 2 Mite Polypeptides.

Group 2 mite polypeptides may be epitope mapped using the proprietary in silico epitope mapping tool disclosed in detail in WO 00/26230 and WO 01/83559. In brief, this tool comprises a database of epitope patterns (determined from an input of peptide sequences, known to bind specifically to anti-protein antibodies) and an algorithm to analyse 3-D structure of a given protein against the epitope pattern database. This will determine the possible epitopes on that protein, and the preference of each amino acid in the protein sequence to be part of epitopes.

Identifying Antibody-Binding Peptides:

Antibody-binding peptides can be identified by many different ways. One is to synthesize a number of peptides of known sequence, and test for their ability to bind antibodies of interest, e.g., in ELISA or other immunochemical assays. Such data are available in great abundance in the literature.

A particularly effective way is to prepare a library of many different random peptide sequences and select experimentally only the ones that bind antibodies well and specific (i.e., can be outcompeted by the protein towards which the antibodies were raised). Phage display techniques are well suited for this way of finding antibody bidning peptides:

In a phage display system, a sequence encoding a desired amino acid sequence is incorporated into a phage gene coding for a protein displayed on the surface of the phage. Thus, the phage will make and display the hybrid protein on its surface, where it can interact with specific target agents. Given that each phage contains codons for one specific sequence of a determined length, an average phage display library can express 10⁸-10¹² different random sequences. If the displayed sequence resembles an epitope, the phage can be selected by an epitope-specific antibody. Thus, it is possible to select specific phages from the bulk of a large number of phages, each expressing their one hybrid protein.

It is important that the amino acid sequence of the (oligo)peptides presented by the phage display system have a sufficient length to present a significant part of an epitope to be identified. The oligopeptides may have from 5 to 25 amino acids, preferably at least 8 amino acids, such as 9 amino acids.

The antibodies used for reacting with the oligopeptides can be polyclonal or monoclonal. In particular, they may be IgE antibodies to ensure that the epitopes identified are IgE epitopes, i.e., epitopes inducing and binding IgE. The antibodies may also be monospecific, meaning they are isolated according to their specificity for a certain protein. Polyclonal antibodies are preferred for building up data on antibody-binding peptides to be used in the in silico mapping tool in order to obtain a broader knowledge about the epitopes of a polypeptide. These reactive peptides, by virtue of their reactivity against antibodies, to some degree resemble the appearance of an epitope on a full polypeptide.

Identifying Epitope Patterns from Reactive Petides

The reactive (oligo)peptides identified e.g. by phage display are compared and aligned in order to identify common epitope patterns, which then can be used for identification of antibody binding epitopes on a 3-dimensional polypeptide.

In the alignment conservative alternatives to an amino acid such as aspartate and glutamate, lysine and arginine, serine and threonine are considered as one or equal.

Thus, the alignment results in a number of patterns, which depend on the chosen number of residues of the peptides. Using for example a 7-mer peptide, the pattern may have the form:
X X * * X X X,

where “*” in this notation indicates a non-consensus moiety which may be any amino acid or group of amino acids or no amino acid, while X is one of the following 13 residue types: AG, C, DE, FY, H, IL, KR, M, NQ, P, ST, V, and W, where the pairs AG, DE, FY, IL, KR, NQ, ST are conservative alternatives and considered equal. Accordingly, 3 peptides such as

A K S N N K R
A K S M N K R
A K T P N K K

would create a pattern of [AG] [KR] [ST]* [NQ] [KR] [KR], where the residues AG KR ST and NQ KR KR are consensus residues shared by all 3 peptides and thus the epitope pattern would be AG KR ST * NQ KR KR. The patterns are chosen to describe a complete set of reactive (oligo)peptides (obtained e.g., by a phage display and antibody reaction) by the fewest possible patterns.

The epitope patterns may be determined directly from the reactive peptides; if for example a library of 7-mer reactive peptides is made, one can use each different reactive 7 mer peptide, taking conservative alternatives into account, as an epitope pattern in the epitope mapping approach as described below.

It is also possible to reduce the number of epitope patterns to be examined in the epitope mapping by removing redundant patterns and/or by employing experimental designs as known in the art (see example 1).

Within the identified epitope patterns some amino acids are conservative, called anchor amino acids. The anchor amino acids recur in all or a majority of the reactive peptides.

Epitope Mapping Algorithm

When epitope patterns have been identified they are subsequently compared to the three-dimensional coordinates of the amino acid sequence of the polypeptide of interest, in order to identify combinations of residues on the polypeptide surface corresponding to the consensus sequence(s) or epitope pattern(s). In this way, amino acids residues, which are important for antibody binding, can be identified.

Once one or more epitope patterns have been identified, any polypeptide for which a three-dimensional structure is known may be analysed for epitopes matching the epitope patterns. Finding an epitope on a polypeptide is achieved by searching the surface of the polypeptide in the following way:
(1) For all amino acids in the polypeptide it is examined if (a) the amino acid type match the first amino acid of an epitope pattern and (b) the surface accessibility greater than or equal to a chosen threshold allowing the amino acid to be immunological interactive. Those amino acid satisfying 1(a) and 1(b) are selected.
(2) For all amino acids within a selected distance (e.g. 10 Angstroms) of the amino acids selected in step 1 it is examined if (a) the amino acid type matches the second amino acid of the pattern and (b) the surface accessibility greater than or equal to a chosen threshold allowing the amino acid to be immunological interactive. Those amino acid satisfying 2(a) and 2(b) are selected
(3) For all amino acids within a selected distance (e.g., 10 Angstroms) of the amino acids selected in step 2 it is examined if (a) the amino acid type matches the third amino acid of the pattern and (b) the surface accessibility greater than or equal to a chosen threshold allowing the amino acid to be immunological interactive. Those amino acid satisfying 3(a) and 3(b) are selected.

This procedure (step 3) is repeated for all amino acids in the epitope pattern consensus sequence. The coordinates of its C-alpha atom define the spatial positioning of an amino acid. The surface solvent accessibility threshold is given in percent of an average for the particular residue type (see example 2).

If matching amino acids for all amino acids in the epitope pattern can be found in the structure of the polypeptide it is a very strong indication that an epitope has been found. However it is also checked that the size of the epitope is satisfactory, i.e., the distance between any two residues is below a given threshold, usually 25 Å.

The epitopes found may be ranked and weighted according to their total accessible surface area, in order to improve further the predictability of the tool.

Finally, when all possible epitopes have been mapped for the protein of interest, one can provide a score for each amino acid of the protein by adding up the number of times it appears in an epitope pattern. This score will be an indication of the likelyhood that modification (substitution, insertion, deletion, glycosylation or chemical conjugation) of that amino acid will, result in a variant with a lower antigenicity. All amino acids of the protein can then be ranked according to this score and those with highest scores can be selected for mutagenesis.

The epitope mapping tool can be adjusted, such that only a subset of the known reactive peptides are included as data set for building epitope patterns, and thus for conducting epitope mapping. For instance, one may choose only to include peptides reactive to IgE antibodies (rather than to IgG or other antibodies), or one may include only peptides reactive to human antibodies etc. One may choose to involve only peptides reactive against the target protein in order to get a more specific response, however, in general, peptides reactive to antibodies that in turn were raised against any protein are included.

If no three-dimensional structure coordinates are available for the protein of interest, one can map the epitope patterns directly to the primary sequence of the protein of interest. From all the above information, it is obvious, that the epitopes are conveniently determined using this epitope mapping tool.

Further, the in silico epitope mapping tool can be used to predict if mutating one amino acid residue will result in that the new variant overall will have fewer epitopes. Thus, some or all 19 possible substitutions can be tested in a given position, the epitope mapping procedure repeated for a model structure of each of these proposed variants, and the best variant(s) can be constructed by mutation and tested experimentally.

Identified Epitopes of Group 2 Mite Polypeptides

Using the epitope mapping tool the present inventors have surprisingly identified epitope areas (EA) in group 2 mite polypeptides of Dermatophagoides pteronyssinus (Der p 2) and Dermatophagoides farinae (Der f 2) which are involved in antibody binding epitopes and for which a mutation have an altering, preferably reducing, effect on the antibody binding, particularly IgE binding of the polypeptide. Hence, the present invention provide in a first aspect a variant of a group 2 mite polypeptide, wherein the polypeptide of the variant comprises one or more mutations in the epitope areas of Der p 2 and one or more mutations in the epitope areas of Der f2.

The IgG epitope areaes at 70% exposion to solvent (EA1), at 60% exposion to solvent (EA2), and at 40% exposion to solvent (EA3 to EA5) of Der p 2 consist of the positions

EA1: N46 K48 T49 P79 D113 D114 G115,

EA2: P26 I28 H30 R31 S57 E102 N103 V105 K126 R128,

EA3: S1 Q2 D4 L17 P19 G20 H22 S24 E25 I28 H30 I97 K100 E102 H124 K126 I127,

EA4: K14 V40 E42 N44 Q85,

EA5: E53 G60 L61 E62 D64 V65 P66 M111,

of the mature Der p 2 polypeptide (SEQ ID No: 1).

The IgE epitope areaes at 70% exposion to solvent (EA6), at 60% exposion to solvent (EA7 and EA8), and at 40% exposion to solvent (EA9 to EA12) of Der p 2 consist of the positions

EA6: N46 K48 P79 V81 D113 D114,

EA7: P19 H22 S24 E25 P26 I28 H30 R31 K126 R128,

EA8: K55 S57 G60 L61 E62 D64 P95 E102 N103 V105 T123,

EA9: S1 Q2 D4 K6 L17 P19 H22 S24 E25 P26 I28 I29 H30 R31 P34 W92 N93 P95 K96 I97 A98 T123 H124,

EA10: E53 K55 S57 G60 L61 E62 V63 D64 P66 K100 E102 N103 V105 T107 K126 R128,

EA11: K14 V40 E42 H74 Y75 M76 K77 P79 V81 K82 G83 Q84 Q85 D87 K89,

EA12: C8 A9 N10 N44 Q45 N46 K48 M111 D113 D114 G115 V116

of the mature Der p 2 polypeptide (SEQ ID No: 1).

The IgG epitope areaes at more than 50% exposion to solvent (EA13 to EA15) of Der f 2 consist of the positions:

EA13: D1 Q2 M17 D19 H30 R31 G32 K33 P34 T91 N93 P95 A98 P99 R128,

EA14: D59 L61 E62 D64 E102 N103 T107 K126,

EA15: N46 T49 C73 F75 N114

of the mature Der f 2 polypeptide (SEQ ID No: 2).

The IgE epitope areaes at more than 50% exposion to solvent (EA16 to EA19) of Der f 2 consist of the positions:

EA16: D1 D4 K6 D7 D19 H22 S24 D25 P26 T123 H124 K126,

EA17: H30 R31 G32 K33 P95 K96 I97 A98 P99 K100 R128,

EA18: D59 L61 E52 T91 N93 G102 N103 V105,

EA19: N46 K48 T49 K51 N71 A72 K82 G83 D113 N114 G115

of the mature Der f 2 polypeptide (SEQ ID No: 2).

Particularly, said variants have reduced IgE-binding, more particularly combined with preserved immunogenicity for inducing protective responses. Still more preferably, the variants have an altered immunogenic profile in exposed animals, including humans, as compared to the native group 2 mite polypeptide.

Group 2 mite polypeptides are highly homologeous and the corresponding positions in Group 2 mite polypeptides of various sources may easily be found by aligning such polypeptides with SEQ ID NO:1.

Group 2 Mite Polypeptide Variants

Selection of Positions for Mutation

Once the epitopes of a polypeptide have been determined, variants of the polypeptide with modified antigenic properties can be made by mutating one or more of the amino acid residues comprised in the epitope. In this context mutation encompasses deletion and/or substitution of an amino acid residue and/or insertion of one or more amino acids before or after that residue.

When providing a polypeptide variant suitable as a vaccine agent, for treatment of allergies, it is particularly desirable to alter IgE epitopes to reduce the binding of IgE, while at the same time maintaining the ability of the variant to invoke an immune response in humans and animals, hence it is desirable that the polypeptides retains the three-dimensional conformation of the parent polypeptide. Further it is particularly desirable that the variant is capable of stimulating T-cells sufficiently, preferable at the level of the parent polypeptide or better. Still further it is desirable that the variant is capable of invoking an IgG response in human and animals.

The epitope identified may be mutated by substituting at least one amino acid of the epitope. In a particular embodiment at least one anchor amino acid is mutated. The mutation will often be a substitution with an amino acid of different size, hydrophilicity, polarity and/or acidity, such as a small amino acid in exchange of a large amino acid, a hydrophilic amino acid in exchange of a hydrophobic amino acid, a polar amino acid in exchange of a non-polar amino acid and a basic in exchange of an acidic amino acid.

Other mutations may be the insertion or deletion of at least one amino acid of the epitope, particularly deleting an anchor amino acid. Furthermore, an epitope may be mutated by substituting some amino acids, and deleting and/or inserting others.

The mutation(s) performed may be performed by standard techniques well known to a person skilled in the art, such as site-directed mutagenesis (see, e.g., Sambrook et al. (1989), Molecular Cloning. A Laboratory Manual, Cold Spring Harbor, N.Y.).

The mutagenesis may be spiked mutagenesis which is a form of site-directed mutagenesis, in which the primers used have been synthesized using mixtures of oligonucleotides at one or more positions.

A general description of nucleotide substitution can be found in e.g., Ford et al., 1991, Protein Expression and Purification 2, pp. 95-107.

The polypeptide variant of the invention concerns variants of parent group 2 mite polypeptides comprising one or more mutations in the parent polypeptide in the positions or corresponding to the positions consisting of V40, E53, S57, D64, K82, G83, and I97 of SEQ ID NO: 1 or 40, 53, 57, 64, 82, 83, and 97 of the mature Der p 2 polypeptide, and to the positions consisting of G32, D59, L61, E62, A98 of SEQ ID NO: 2 or 32, 59, 61, 62, 98 of the Der f 2 polypeptide.

The above identified mutations (V40, E53, S57, D64, K82, G83, and I97 of SEQ ID NO: 1 and G32, D59, L61, E62, A98 of SEQ ID NO: 2) or combinations thereof may preferably be introduced to the group 2 mite polypeptide simultanously with one or more mutations corresponding to positions identified in EA1 to EA12 for Der p 2 and in EA13 to EA19 for Der f2.

Particularly interesting variants of SEQ ID NO: 1 are variants comprising mutations in positions corresponding to V40, E53, S57, D64, K82, G83, and I197 of SEQ ID NO: 1, or combinations hereof, in combination with mutations corresponding to N46, K48, T49, P79, V81, D113, D114, G115 of SEQ ID NO: 1.

Particularly interesting variants of SEQ ID NO: 2 are variants comprising mutations in positions corresponding to G32, D59, L61, E62, A98 of SEQ ID NO: 2, or combinations hereof, in combination with mutations corresponding to D19, R31, K33, N46, T49, D59, and N114 of SEQ ID NO: 2.

Particularly the variant has an altered antibody binding profile as compared the parent group 2 mite polypeptide, more particularly the variant have a reduced IgE-binding, more particularly combined with preserved immunogenicity for inducing protective responses. Still more preferably, the variant have an altered immunogenic profile in exposed animals, including humans, as compared to the parent group 2 mite polypeptide.

Further the variant have in particular an altered IgE-antigenicity as compared to the parent group 2 mite polypeptide.

Still further the variant have in particular at least the same T-cell stimulatory effect compared to the parent group 2 mite polypeptide as measured by the procedure in example 6. Still further the variant induces an altered immunogenic response in exposed animals, including humans, as compared to the parent group 2 mite polypeptide.

Still further the variant induces in particular an altered immunogenic response in humans, as compared to the parent group 2 mite polypeptide.

In a particular embodiment the group 2 mite polypeptide has in its mature form a sequence which displays at least 80% identity to SEQ ID NO:3; in particular, at least 85% identity, in particular at least 90% identity; in particular at least 95% identity, more particularly 98% identity, more particularly 100% identity to SEQ ID NO:3 or 100% identity to Euroglyphus maynei 2 (Eur m 2).

Mutations Directly Providing for Reduced Antigenicity.

The risk linked to protein engineering in order to eliminate epitopes that new epitopes are made, or existing epitopes are duplicated is reduced by testing the planned mutations at a given position in the 3-dimensional structure of the protein of interest against the found epitope patterns thereby identifying the mutations for each position that are feasible for obtaining the desired properties of the polypeptide.

In a particular embodiment of the invention, it will be an advantage, to establish a library of diversified mutants each having one or more changed amino acids introduced and selecting those variants, which show the best effect as vaccine agent while the fewest side effects. A diversified library can be established by a range of techniques known to the person skilled in the art (Reetz M T; Jaeger K E, in Biocatalysis—from Discovery to Application edited by Fessner W D, Vol. 200, pp. 31-57 (1999); Stemmer, Nature, vol. 370, p. 389-391, 1994; Zhao and Arnold, Proc. Natl. Acad. Sci., USA, vol. 94, pp. 7997-8000, 1997; or Yano et al., Proc. Natl. Acad. Sci., USA, vol. 95, pp 5511-5515, 1998). In a more preferable embodiment, substitutions are found by a method comprising the following steps: 1) a range of substitutions, additions, and/or deletions are listed encompassing several epitopes, 2) a library is designed which introduces a randomized subset of these changes in the amino acid sequence into the target gene, e.g., by spiked mutagenesis, 3) the library is expressed, and preferred variants are selected. In a most preferred embodiment, this method is supplemented with additonal rounds of screening and/or family shuffling of hits from the first round of screening (J. E. Ness, et al, Nature Biotechnology, vol. 17, pp. 893-896, 1999).

Mutations Providing for Increased Glycosylation to Amino Acids in the Epitope Area.

In another approach, the mutations are designed, such that recognition sites for post-translational modifications are introduced in the epitope areas, and the protein variant is expressed in a suitable host organism capable of the corresponding post-translational modification. These post-translational modifications may serve to shield the epitope and hence lower the immunogenicity of the protein variant relative to the protein backbone. Post-translational modifications include glycosylation, phosphorylation, N-terminal processing, acylation, ribosylation and sulfatation. A good example is N-glycosylation. N-glycosylation is found at sites of the sequence Asn-Xaa-Ser, Asn-Xaa-Thr, or Asn-Xaa-Cys, in which neither the Xaa residue nor the amino acid following the tri-peptide consensus sequence is a proline (T. E. Creighton, ‘Proteins—Structures and Molecular Properties, 2nd edition, W.H. Freeman and Co., New York, 1993, pp. 91-93). It is thus desirable to introduce such recognition sites in the sequence of the backbone protein. The specific nature of the glycosyl chain of the glycosylated protein variant may be linear or branched depending on the protein and the host cells. Another example is phosphorylation: The protein sequence can be modified so as to introduce serine phophorylation sites with the recognition sequence arg-arg-(xaa)ₙ-ser (where n=0, 1, or 2), which can be phosphorylated by the cAMP-dependent kinase or tyrosine phosphorylation sites with the recognition sequence -lys/arg-(xaa)₃-asp/glu-(xaa)₃-tyr, which can usually be phophorylated by tyrosine-specific kinases (T. E. Creighton, “Proteins—Structures and molecular properties”, 2nd ed., Freeman, NY, 1993).

Mutations Providing for Covalent Conjugation of Polymers to Amino Acids in the Epitope Area.

Another way of making mutations that will change the antigenic properties of a polypeptide is to react or conjugate polymers to amino acids in or near the epitope, thus blocking or shielding the access to the anchor amino acids and thus the binding of antibodies and/or recptors to those amino acids. If no amino acid suitable for conjugation with a polymer exists in the parent polypeptide a suitable mutation is the insertion of one or more amino acids being attachment sites and/or groups and/or amino acids for polymer conjugation.

Which amino acids to substitute and/or insert depends in principle on the coupling chemistry to be applied. The chemistry for preparation of covalent bioconjugates can be found in “Bioconjugate Techniques”, Hermanson, G. T. (1996), Academic Press Inc., which is hereby incorporated as reference.

It a particular embodiment activated polymers are conjugated to amino acids in or near the epitope area.

It is preferred to make conservative substitutions in the polypeptide when the polypeptide has to be conjugated, as conservative substitutions secure that the impact of the substitution on the polypeptide structure is limited.

In the case of providing additional amino groups, this may be done by substitution of Arginine to Lysine, both residues being positively charged, but only the Lysine having a free amino group suitable as an attachment groups.

In the case of providing additional carboxylic acid groups, the conservative substitution may for instance be an Asparagine to Aspartic acid or Glutamine to Glutamic acid substitution. These residues resemble each other in size and shape, except from the carboxylic groups being present on the acidic residues.

In the case of providing SH-groups the conservative substitution may be done by substitution of Threonine or Serine to Cysteine.

Verification of Variants Having Altered Antigenic Properties

The mutation of amino acids, comprised in an epitope, will cause the antigenic properties of the polypeptide to change, as predicted by the in silico determination of the epitopes. However, the quantitative effect of the mutation on the antigenicity, i.e., the antibodybinding, and the immunogenicity of the variant, is suitably determined using various in vivo or in vitro model systems. For that use, the polypeptide variant of interest can be expressed in larger scale and purified by conventional techniques. Then the functionality and specific activity may be tested by cysteine protease activity assays, in order to assure that the variant has retained three-dimensional structure.

In vitro systems include assays measuring binding to IgE in serum from dust mite allergic patients or exposed animals, cytokine expression profiles or proliferation responses of T-cells from dust mite allergic patients or exposed animals, and histamine release from basophils from dust mite allergic patients.

The IgE antibody binding can be examined in detail using, e.g., direct or competitive ELISA (C-ELISA), histamine release assays on basophil cells from allergic patients, or IgE-stripped basophils from cord blood incubated with IgE-containing serum from allergic patients, or by other or other solid phase immunoassays or cellular assays (see example 5).

In a particular embodiment the ability of the polypeptide variant to bind IgE is reduced at least 3 times as compared to the binding ability of the orginal or parent group 2 mite polypeptide, preferably 10 times reduced, more preferably 50 times.

In a further particular embodiment the ability of the polypeptide variant to induce histamine release in basophil cells from subjects allergic to dust mites is reduced least 3 times, as compared to that of the parent group 2 mite polypeptide, preferably 10 times reduced, more preferably 50 times.

In a further embodiment, the ability of the polypeptide variant to invoke a recall T-cell response in lymphocytes from animals, including humans, previously exposed to the orginal or parent group 2 mite allergen is measured, preferably the strength of the response is comparable to or higher than that to the parent group 2 mite allergen (see example 6).

In a particular embodiment the in vivo verification comprises skin prick testing (SPT), in which a dust mite allergic subject/indvidual is exposed to intradermal or subcutaneous injection of group 2 mite polypeptides and the IgE reactivity, measured as the diameter of the wheal and flare reaction, in response to a polypeptide variant of the invention is compared to that to the parent group 2 mite polypeptide (Kronquist et al., Clin. Exp. Allergy, 2000, vol. 30, pp. 670-676).

The in vivo immunogenic properties of the polypeptide variant of the invention may also suitably be measured in an animal test, wherein test animals are exposed to a parent group 2 mite polypeptide and the responses to variants as well as to the parent group 2 allergen are measured. The immune response measurements may include comparing reactivity of serum IgE or T-cells from a test animal with a parent group 2 mite polypeptide and the polypeptide variant.

In a particular embodiment the in vivo verification comprises exposing a mouse to a parent group 2 mite polypeptide by the intranasal route, and verifying that serum IgE is less reactive with a polypeptide variant than with the parent group 2 mite polypeptide. Useful in vivo animal models include the mouse intranasal test (MINT) model (Robinson et al., Fund. Appl. Toxicol. 34, pp. 15-24, 1996).

In a further particular embodiment the in vivo verification comprises exposing a test animal to a polypeptide variant by the intratracheal route and verifying that the specific IgE titers are lower than with the parent group 2 mite polypeptide. Useful in vivo animal models include the guinea pig intratracheal (GPIT) model (Ritz, et al. Fund. Appl. Toxicol., 21, pp. 31-37, 1993) and the rat intratracheal (rat-IT) model (WO 96/17929, Novo Nordisk).

In a further particular embodiment, the in vivo verification comprises exposing a test animal subcutaneously to the parent group 2 allergen and the polypeptide variant, and verifying that T cell reactivity and cross-reactivity is comparable. Also, IgE binding and cross reactivity can be measured following this route of exposure. A suitable model is the mouse subcutaneous (mouse-SC) model (WO 98/30682, Novo Nordisk).

Preparation of Nucleotide Constructs, Vectors, Host Cells, Protein Variants and Polymers for Conjugation.

In accordance with the present invention there may be employed conventional molecular biology, microbiology, and recombinant DNA techniques well known to a person skilled in the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”) DNA Cloning: A Practical Approach, Volumes I and II/D. N. Glover ed. 1985); Oligonucleotide Synthesis (M. J. Gait ed. 1984); Nucleic Acid Hybridization (B. D. Hames & S. J. Higgins eds (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984).

A preferred method is to express the group 2 dust mite proteins in S. cerevisiae cells, as described by Hakkaart et al. (Clin. and Exper. Allergy 1998, vol. 28, pp 45-52).

Another preferred method is to express group 2 dust mite proteins in E. coli (Mueller et al, J. Biol. Chem. 1997, vol. 272, p 26893-26898).

Nucleotide Sequences

The present invention also encompasses a nucleotide sequence encoding a polypeptide variant of the invention. As described, a description of standard mutation of nucleotide sequences to encode polypeptide variants by nucleotide substitution can be found in e.g., Ford et al., 1991, Protein Expression and Purification 2, p. 95-107. Other standard methods, such as site-directed mutagenesis is described in e.g., Sambrook et al. (1989), Molecular Cloning. A Laboratory Manual, Cold Spring Harbor, N.Y.

A “nucleotide sequence” is a single- or double-stranded polymer of deoxyribonucleotide or ribonucleotide bases read from the 5′ to the 3′ end. Nucleotide sequences include RNA and DNA, and may be isolated from natural sources, synthesized in vitro, or prepared from a combination of natural and synthetic molecules.

The techniques used to isolate or clone a nucleotide sequence alias a nucleotide sequence encoding a polypeptide are known in the art and include isolation from genomic DNA, preparation from cDNA, or a combination thereof. The cloning of the nucleotide sequences of the present invention from such genomic DNA can be effected, e.g., by using the well known polymerase chain reaction (PCR) or antibody screening of expression libraries to detect cloned DNA fragments with shared structural features. See, e.g. Innis et al., 1990, A Guide to Methods and Application, Academic Press, New York. Other nucleotide amplification procedures such as ligase chain reaction (LCR), ligated activated transcription (LAT) and nuceic acid sequence-based amplification (NASBA) may be used. The nucleotide sequence may be cloned from a strain producing the polypeptide, or from another related organism and thus, for example, may be an allelic or species variant of the polypeptide encoding region of the nucleotide sequence.

The term “isolated” nucleotide sequence as used herein refers to a nucleotide sequence which is essentially free of other nucleotide sequences, e.g., at least about 20% pure, preferably at least about 40% pure, more preferably about 60% pure, even more preferably about 80% pure, most preferably about 90% pure, and even most preferably about 95% pure, as determined by agarose gel electorphoresis. For example, an isolated nucleotide sequence can be obtained by standard cloning procedures used in genetic engineering to relocate the nucleotide sequence from its natural location to a different site where it will be reproduced. The cloning procedures may involve excision and isolation of a desired nucleotide fragment comprising the nucleotide sequence encoding the polypeptide, insertion of the fragment into a vector molecule, and incorporation of the recombinant vector into a host cell where multiple copies or clones of the nucleotide sequence will be replicated. The nucleotide sequence may be of genomic, cDNA, RNA, semisynthetic, synthetic origin, or any combinations thereof. Such isolated molecules are those that are separated from their natural environment and include cDNA and genomic clones. Isolated DNA molecules of the present invention are free of other genes with which they are ordinarily associated, and may include naturally occurring 5′ and 3′ untranslated regions such as promoters and terminators. The identification of associated regions will be evident to one of ordinary skill in the art (see for example, Dynan and Tijan, Nature 316: 774-78, 1985).

Nucleotide Construct

As used herein the term “nucleotide construct” is intended to indicate any nucleotide molecule of cDNA, genomic DNA, synthetic DNA or RNA origin. The term “construct” is intended to indicate a nucleotide segment which may be single- or double-stranded, and which may be based on a complete or partial naturally occurring nucleotide sequence encoding a polypeptide of interest. The construct may optionally contain other nucleotide segments.

The DNA of interest may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., supra).

The nucleotide construct may also be prepared synthetically by established standard methods, e.g., the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g., in an automatic DNA synthesizer, purified, annealed, ligated and cloned in suitable vectors.

Furthermore, the nucleotide construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleotide construct, in accordance with standard techniques.

The nucleotide construct may also be prepared by polymerase chain reaction using specific primers, for instance as described in U.S. Pat. No. 4,683,202 or Saiki et al., Science 239 (1988), 487-491.

The term nucleotide construct may be synonymous with the term expression cassette when the nucleotide construct contains all the control sequences required for expression of a coding sequence of the present invention.

The term “coding sequence” as defined herein is a sequence which is transcribed into mRNA and translated into a polypeptide of the present invention when placed under the control of the above mentioned control sequences. The boundaries of the coding sequence are generally determined by a translation start codon ATG at the 5′-terminus and a translation stop codon at the 3′-terminus. A coding sequence can include, but is not limited to, DNA, cDNA, and recombinant nucleotide sequences.

The term “control sequences” is defined herein to include all components which are necessary or advantageous for expression of the coding sequence of the nucleotide sequence. Each control sequence may be native or foreign to the nucleotide sequence encoding the polypeptide. Such control sequences include, but are not limited to, a leader, a polyadenylation sequence, a propeptide sequence, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

The nucleotide constructs of the present invention may also comprise one or more nucleotide sequences which encode one or more factors that are advantageous in the expression of the polypeptide, e.g., an activator (e.g., a trans-acting factor), a chaperone, and a processing protease. Any factor that is functional in the host cell of choice may be used in the present invention. The nucleotides encoding one or more of these factors are not necessarily in tandem with the nucleotide sequence encoding the polypeptide.

Propeptides

The control sequence may also be a propeptide coding region, which codes for an amino acid sequence positioned at the amino terminus of a polypeptide. The resultant polypeptide is known as a proenzyme or propolypeptide (or a zymogen in some cases). A propolypeptide is generally inactive and can be converted to mature active polypeptide by catalytic or autocatalytic cleavage of the propeptide from the propolypeptide. The propeptide coding region may be obtained from the Bacillus subtilis alkaline protease gene (aprE), the Bacillus subtilis neutral protease gene (nprT), the Saccharomyces cerevisiae alpha-factor gene, or the Myceliophthora thermophilum laccase gene (WO 95/33836).

Activators

An activator is a protein which activates transcription of a nucleotide sequence encoding a polypeptide (Kudla et al., 1990, EMBO Journal 9:1355-1364; Jarai and Buxton, 1994, Current Genetics 26:2238-244; Verdier, 1990, Yeast 6:271-297). The nucleotide sequence encoding an activator may be obtained from the genes encoding Bacillus stearothermophilus NprA (nprA), Saccharomyces cerevisiae heme activator protein 1 (hap1), Saccharomyces cerevisiae galactose metabolizing protein 4 (gal4), and Aspergillus nidulans ammonia regulation protein (areA). For further examples, see Verdier, 1990, supra and MacKenzie et al., 1993, Journal of General Microbiology 139:2295-2307.

Chaperones

A chaperone is a protein which assists another polypeptide in folding properly (Hartl et al., 1994, TIBS 19:20-25; Bergeron et al., 1994, TIBS 19:124-128; Demolder et al., 1994, Journal of Biotechnology 32:179-189; Craig, 1993, Science 260:1902-1903; Gething and Sambrook, 1992, Nature 355:33-45; Puig and Gilbert, 1994, Journal of Biological Chemistry 269:7764-7771; Wang and Tsou, 1993, The FASEB Journal 7:1515-11157; Robinson et al., 1994, Bio/Technology 1:381-384). The nucleotide sequence encoding a chaperone may be obtained from the genes encoding Bacillus subtilis GroE proteins, Aspergillus oryzae protein disulphide isomerase, Saccharomyces cerevisiae calnexin, Saccharomyces cerevisiae BiP/GRP78, and Saccharomyces cerevisiae Hsp70. For further examples, see Gething and Sambrook, 1992, supra, and Hartl et al., 1994, supra.

Processing Protease

A processing protease is a protease that cleaves a propeptide to generate a mature biochemically active polypeptide (Enderlin and Ogrydziak, 1994, Yeast 10:67-79; Fuller et al., 1989, Proceedings of the National Academy of Sciences USA 86:1434-1438; Julius et al., 1984, Cell 37:1075-1089; Julius et al., 1983, Cell 32:839-852). The nucleotide sequence encoding a processing protease may be obtained from the genes encoding Aspergillus niger Kex2, Saccharomyces cerevisiae dipeptidylaminopeptidase, Saccharomyces cerevisiae Kex2, and Yarrowia lipolytica dibasic processing endoprotease (xpr6).

Promoters

The control sequence may be an appropriate promoter sequence, a nucleotide sequence which is recognized by a host cell for expression of the nucleotide sequence. The promoter sequence contains transcription and translation control sequences which mediate the expression of the polypeptide. The promoter may be any nucleotide sequence which shows transcriptional activity in the host cell of choice and may be obtained from genes encoding extracellular or intracellular polypeptides either homologous or heterologous to the host cell.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

Examples of suitable promoters for directing the transcription of the nucleotide constructs of the present invention, especially in a bacterial host cell, are the promoters obtained from the E. coli lac operon, the Streptomyces coelicolor agarase gene (dagA), the Bacillus subtilis levansucrase gene (sacB), the Bacillus subtilis alkaline protease gene, the Bacillus licheniformis alpha-amylase gene (amyL), the Bacillus stearothermophilus maltogenic amylase gene (amyM), the Bacillus amyloliquefaciens alpha-amylase gene (amyQ), the Bacillus amyloliquefaciens BAN amylase gene, the Bacillus licheniformis penicillinase gene (penP), the Bacillus subtilis xylA and xylB genes, and the prokaryotic beta-lactamase gene (Villa-Kamaroff et al., 1978, Proceedings of the National Academy of Sciences USA 75:3727-3731), as well as the tac promoter (DeBoer et al., 1983, Proceedings of the National Academy of Sciences USA 80:21-25), or the Bacillus pumilus xylosidase gene, or by the phage Lambda PR or PL promoters or the E. coli lac, trp or tac promoters. Further promoters are described in “Useful proteins from recombinant bacteria” in Scientific American, 1980, 242:74-94; and in Sambrook et al., 1989, supra.

Examples of suitable promoters for directing the transcription of the nucleotide constructs of the present invention in a filamentous fungal host cell are promoters obtained from the genes encoding Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium oxysporum trypsin-like protease (as described in U.S. Pat. No. 4,288,627, which is incorporated herein by reference), and hybrids thereof. Particularly preferred promoters for use in filamentous fungal host cells are the TAKA amylase, NA2-tpi (a hybrid of the promoters from the genes encoding Aspergillus niger neutral (-amylase and Aspergillus oryzae triose phosphate isomerase), and glaA promoters. Further suitable promoters for use in filamentous fungus host cells are the ADH3 promoter (McKnight et al., The EMBO J. 4 (1985), 2093-2099) or the tpiA promoter.

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH24c (Russell et al., Nature 304 (1983), 652-654) promoters.

Further useful promoters are obtained from the Saccharomyces cerevisiae enolase (ENO-1) gene, the Saccharomyces cerevisiae galactokinase gene (GAL1), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene. Other useful promoters for yeast host cells are described by Romanos et al., 1992, Yeast 8:423-488. In a mammalian host cell, useful promoters include viral promoters such as those from Simian Virus 40 (SV40), Rous sarcoma virus (RSV), adenovirus, and bovine papilloma virus (BPV).

Examples of suitable promoters for directing the transcription of the DNA encoding the polypeptide of the invention in mammalian cells are the SV40 promoter (Subramani et al., Mol. Cell Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Terminators

The control sequence may also be a suitable transcription terminator sequence, a sequence recognized by a host cell to terminate transcription. The terminator sequence is operably linked to the 3′ terminus of the nucleotide sequence encoding the polypeptide. Any terminator which is functional in the host cell of choice may be used in the present invention. Preferred terminators for filamentous fungal host cells are obtained from the genes encoding Aspergillus oryzae TAKA amylase, Aspergillus niger glucoamylase, Aspergillus nidulans anthranilate synthase, Aspergillus niger alpha-glucosidase, and Fusarium oxysporum trypsin-like protease for fungal hosts) the TPI1 (Alber and Kawasaki, op. cit.) or ADH3 (McKnight et al., op. cit.) terminators.

Preferred terminators for yeast host cells are obtained from the genes encoding Saccharomyces cerevisiae enolase, Saccharomyces cerevisiae cytochrome C (CYC1), or Saccharomyces cerevisiae glyceraldehyde-3-phosphate dehydrogenase. Other useful terminators for yeast host cells are described by Romanos et al., 1992, supra.

Polyadenylation Signals

The control sequence may also be a polyadenylation sequence, a sequence which is operably linked to the 3′ terminus of the nucleotide sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes encoding Aspergillus oryzae TAKA amylase, Aspergillus niger glucoamylase, Aspergillus nidulans anthranilate synthase, and Aspergillus niger alpha-glucosidase.

Useful polyadenylation sequences for yeast host cells are described by Guo and Sherman, 1995, Molecular Cellular Biology 15:5983-5990.

Polyadenylation sequences are well known in the art for mammalian host cells such as SV40 or the adenovirus 5 E1b region.

Signal Sequences

The control sequence may also be a signal peptide coding region, which codes for an amino acid sequence linked to the amino terminus of the polypeptide which can direct the expressed polypeptide into the cell's secretory pathway of the host cell. The 5′ end of the coding sequence of the nucleotide sequence may inherently contain a signal peptide coding region naturally linked in translation reading frame with the segment of the coding region which encodes the secreted polypeptide. Alternatively, the 5′ end of the coding sequence may contain a signal peptide coding region which is foreign to that portion of the coding sequence which encodes the secreted polypeptide. A foreign signal peptide coding region may be required where the coding sequence does not normally contain a signal peptide coding region. Alternatively, the foreign signal peptide coding region may simply replace the natural signal peptide coding region in order to obtain enhanced secretion relative to the natural signal peptide coding region normally associated with the coding sequence. The signal peptide coding region may be obtained from a glucoamylase or an amylase gene from an Aspergillus species, a lipase or proteinase gene from a Rhizomucor species, the gene for the alpha-factor from Saccharomyces cerevisiae, an amylase or a protease gene from a Bacillus species, or the calf preprochymosin gene. However, any signal peptide coding region capable of directing the expressed polypeptide into the secretory pathway of a host cell of choice may be used in the present invention.

A “secretory signal sequence” is a DNA sequence that encodes a polypeptide (a “secretory peptide” that, as a component of a larger polypeptide, directs the larger polypeptide through a secretory pathway of a cell in which it is synthesized. The larger polypeptide is commonly cleaved to remove the secretory peptide during transit through the secretory pathway.

An effective signal peptide coding region for bacterial host cells is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprS, nprM), and the Bacillus subtilis PrsA gene. Further signal peptides are described by Simonen and Palva, 1993, Microbiological Reviews 57:109-137.

An effective signal peptide coding region for filamentous fungal host cells is the signal peptide coding region obtained from Aspergillus oryzae TAKA amylase gene, Aspergillus niger neutral amylase gene, the Rhizomucor miehei aspartic proteinase gene, the Humicola lanuginosa cellulase or lipase gene, or the Rhizomucor miehei lipase or protease gene, Aspergillus sp. amylase or glucoamylase, a gene encoding a Rhizomucor miehei lipase or protease. The signal peptide is preferably derived from a gene encoding A. oryzae TAKA amylase, A. niger neutral (-amylase, A. niger acid-stable amylase, or A. niger glucoamylase.

Useful signal peptides for yeast host cells are obtained from the genes for Saccharomyces cerevisiae a-factor and Saccharomyces cerevisiae invertase. Other useful signal peptide coding regions are described by Romanos et al., 1992, supra.

For secretion from yeast cells, the secretory signal sequence may encode any signal peptide which ensures efficient direction of the expressed polypeptide into the secretory pathway of the cell. The signal peptide may be a naturally occurring signal peptide, or a functional part thereof, or it may be a synthetic peptide. Suitable signal peptides have been found to be the a-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and uptream of the DNA sequence encoding the polypeptide. The function of the leader peptide is to allow the expressed polypeptide to be directed from the endoplasmic reticulum to the Golgi apparatus and further to a secretory vesicle for secretion into the culture medium (i.e. exportation of the polypeptide across the cell wall or at least through the cellular membrane into the periplasmic space of the yeast cell). The leader peptide may be the yeast a-factor leader (the use of which is described in e.g., U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

Other Regulator Sequences

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems would include the lac, tac, and trp operator systems. In yeast, the ADH2 system or GAL1 system may be used. In filamentous fungi, the TAKA alpha-amylase promoter, Aspergillus niger glucoamylase promoter, and the Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleotide sequence encoding the polypeptide would be placed in tandem with the regulatory sequence.

Recombinant Expression Vector Comprising Nucleotide Construct

The present invention also relates to a recombinant expression vector comprising a nucleotide sequence of the present invention, a promoter, and transcriptional and translational stop signals. The various nucleotide and control sequences described above may be joined together to produce a recombinant expression vector which may include one or more convenient restriction sites to allow for insertion or substitution of the nucleotide sequence encoding the polypeptide at such sites. Alternatively, the nucleotide sequence of the present invention may be expressed by inserting the nucleotide sequence or a nucleotide construct comprising the sequence into an appropriate vector for expression. In creating the expression vector, the coding sequence is located in the vector so that the coding sequence is operably linked with the appropriate control sequences for expression, and possibly secretion.

“Operably linked”, when referring to DNA segments, indicates that the segments are arranged so that they function in concert for their intended purposes, e.g., transcription initiates in the promoter and proceeds through the coding segment to the terminator.

An “Expression vector” is a DNA molecule, linear or circular, that comprises a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both.

The recombinant expression vector may be any vector (e.g., a plasmid or virus), which can be conveniently subjected to recombinant DNA procedures and can bring about the expression of the nucleotide sequence. The choice of the vector will typically depend on the compatibility of the vector with the host cell into which the vector is to be introduced. The vectors may be linear or closed circular plasmids. The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. The vector system may be a single vector or plasmid or two or more vectors or plasmids which together contain the total DNA to be introduced into the genome of the host cell, or a transposon.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol, tetracycline, neomycin, hygromycin or methotrexate resistance. A frequently used mammalian marker is the dihydrofolate reductase gene (DHFR). Suitable markers for yeast host cells are ADE2, HIS3, LEU2, LYS2, MET3, TRP1, and URA3. A selectable marker for use in a filamentous fungal host cell may be selected from the group including, but not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), and glufosinate resistance markers, as well as equivalents from other species. Preferred for use in an Aspergillus cell are the amdS and pyrG markers of Aspergillus nidulans or Aspergillus oryzae and the bar marker of Streptomyces hygroscopicus. Furthermore, selection may be accomplished by co-transformation, e.g., as described in WO 91/17243, where the selectable marker is on a separate vector.

The vectors of the present invention preferably contain an element(s) that permits stable integration of the vector into the host cell genome or autonomous replication of the vector in the cell independent of the genome of the cell.

The vectors of the present invention may be integrated into the host cell genome when introduced into a host cell. For integration, the vector may rely on the nucleotide sequence encoding the polypeptide or any other element of the vector for stable integration of the vector into the genome by homologous or nonhomologous recombination. Alternatively, the vector may contain additional nucleotide sequences for directing integration by homologous recombination into the genome of the host cell. The additional nucleotide sequences enable the vector to be integrated into the host cell genome at a precise location(s) in the chromosome(s). To increase the likelihood of integration at a precise location, the integrational elements should preferably contain a sufficient number of nucleotides, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination. These nucleotide sequences may be any sequence that is homologous with a target sequence in the genome of the host cell, and, furthermore, may be non-encoding or encoding sequences.

For autonomous replication, the vector may further comprise an origin of replication enabling the vector to replicate autonomously in the host cell in question. Examples of bacterial origins of replication are the origins of replication of plasmids pBR322, pUC19, pACYC177, pACYC184, pUB110, pE194, pTA1060, and pAMβ1. Examples of origin of replications for use in a yeast host cell are the 2 micron origin of replication, the combination of CEN6 and ARS4, and the combination of CEN3 and ARS1. The origin of replication may be one having a mutation which makes its functioning temperature-sensitive in the host cell (see, e.g., Ehrlich, 1978, Proceedings of the National Academy of Sciences USA 75:1433).

More than one copy of a nucleotide sequence encoding a polypeptide of the present invention may be inserted into the host cell to amplify expression of the nucleotide sequence. Stable amplification of the nucleotide sequence can be obtained by integrating at least one additional copy of the sequence into the host cell genome using methods well known in the art and selecting for transformants.

The procedures used to ligate the elements described above to construct the recombinant expression vectors of the present invention are well known to one skilled in the art (see, e.g., Sambrook et al., 1989, supra).

Host Cell Comprising Nucleotide Constructs

The present invention also relates to recombinant host cells, comprising a nucleotide sequence or nucleotide construct or recombinant expression vector of the invention, which are advantageously used in the recombinant production of the polypeptide variants of the invention. The term “host cell” encompasses a parent host cell and any progeny thereof, which is not identical to the parent host cell due to mutations that occur during replication.

The cell is preferably transformed with a vector comprising a nucleotide sequence of the invention followed by integration of the vector into the host chromosome. “Transformation” means introducing a vector comprising a nucleotide sequence of the present invention into a host cell so that the vector is maintained as a chromosomal integrant or as a self-replicating extra-chromosomal vector. Integration is generally considered to be an advantage as the nucleotide sequence is more likely to be stably maintained in the cell. Integration of the vector into the host chromosome may occur by homologous or non-homologous recombination as described above.

The choice of a host cell will to a large extent depend upon the gene encoding the polypeptide and its source. The host cell may be a unicellular microorganism, e.g., a prokaryote, or a non-unicellular microorganism, e.g., a eukaryote. Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a preferred embodiment, the bacterial host cell is a Bacillus lentus, Bacillus licheniformis, Bacillus stearothermophilus or Bacillus subtilis cell. The transformation of a bacterial host cell may, for instance, be effected by protoplast transformation (see, e.g., Chang and Cohen, 1979, Molecular General Genetics 168:111-115), by using competent cells (see, e.g., Young and Spizizin, 1961, Journal of Bacteriology 81:823-829, or Dubnar and Davidoff-Abelson, 1971, Journal of Molecular Biology 56:209-221), by electroporation (see, e.g., Shigekawa and Dower, 1988, Biotechniques 6:742-751), or by conjugation (see, e.g., Koehler and Thorne, 1987, Journal of Bacteriology 169:5771-5278).

The host cell may be a eukaryote, such as a mammalian cell, an insect cell, a plant cell or a fungal cell.

Useful mammalian cells include Chinese hamster ovary (CHO) cells, HeLa cells, baby hamster kidney (BHK) cells, COS cells, or any number of other immortalized cell lines available, e.g., from the American Type Culture Collection.

Examples of suitable mammalian cell lines are the COS (ATCC CRL 1650 and 1651), BHK (ATCC CRL 1632, 10314 and 1573, ATCC CCL 10), CHL (ATCC CCL39) or CHO (ATCC CCL 61) cell lines. Methods of transfecting mammalian cells and expressing DNA sequences introduced in the cells are described in e.g., Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Ausubel et al., Current Protocols in Molecular Biology, John Wiley and Sons, Inc., N.Y., 1987, Hawley-Nelson et al., Focus 15 (1993), 73; Ciccarone et al., Focus 15 (1993), 80; Graham and van der Eb, Virology 52 (1973), 456; and Neumann et al., EMBO J. 1 (1982), 841-845.

In a preferred embodiment, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra). Representative groups of Ascomycota include, e.g., Neurospora, Eupenicillium (=Penicillium), Emericella (=Aspergillus), Eurotium (=Aspergillus), and the true yeasts listed above. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g., Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

In a preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g., genera Pichia, Kluyveromyces and Saccharomyces). The basidiosporogenous yeasts include the genera Leucosporidim, Rhodosporidium, Sporidiobolus, Filobasidium, and Filobasidiella. Yeast belonging to the Fungi Imperfecti are divided into two families, Sporobolomycetaceae (e.g., genera Sorobolomyces and Bullera) and Cryptococcaceae (e.g., genus Candida). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980. The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathern et al., editors, 1981).

The yeast host cell may be selected from a cell of a species of Candida, Kluyveromyces, Saccharomyces, Schizosaccharomyces, Candida, Pichia, Hansehula, or Yarrowia. In a preferred embodiment, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. Other useful yeast host cells are a Kluyveromyces lactis Kluyveromyces fragilis Hansehula polymorpha, Pichia pastoris Yarrowia lipolytica, Schizosaccharomyces pombe, Ustilgo maylis, Candida maltose, Pichia guillermondii and Pichia methanolio cell (cf. Gleeson et al., J. Gen. Microbiol. 132, 1986, pp. 3459-3465; U.S. Pat. No. 4,882,279 and U.S. Pat. No. 4,879,231).

In a preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a vegetative mycelium composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In a more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma or a teleomorph or synonym thereof. In an even more preferred embodiment, the filamentous fungal host cell is an Aspergillus cell. In another even more preferred embodiment, the filamentous fungal host cell is an Acremonium cell. In another even more preferred embodiment, the filamentous fungal host cell is a Fusarium cell. In another even more preferred embodiment, the filamentous fungal host cell is a Humicola cell. In another even more preferred embodiment, the filamentous fungal host cell is a Mucor cell. In another even more preferred embodiment, the filamentous fungal host cell is a Myceliophthora cell. In another even more preferred embodiment, the filamentous fungal host cell is a Neurospora cell. In another even more preferred embodiment, the filamentous fungal host cell is a Penicillium cell. In another even more preferred embodiment, the filamentous fungal host cell is a Thielavia cell. In another even more preferred embodiment, the filamentous fungal host cell is a Tolypocladium cell. In another even more preferred embodiment, the filamentous fungal host cell is a Trichoderma cell. In a most preferred embodiment, the filamentous fungal host cell is an Aspergillus awamori, Aspergillus foetidus, Aspergillus japonicus, Aspergillus niger, Aspergillus nidulans or Aspergillus oryzae cell. In another most preferred embodiment, the filamentous fungal host cell is a Fusarium cell of the section Discolor (also known as the section Fusarium). For example, the filamentous fungal parent cell may be a Fusarium bactridioides, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sulphureum, or Fusarium trichothecioides cell. In another prefered embodiment, the filamentous fungal parent cell is a Fusarium strain of the section Elegans, e.g., Fusarium oxysporum. In another most preferred embodiment, the filamentous fungal host cell is a Humicola insolens or Humicola lanuginosa cell. In another most preferred embodiment, the filamentous fungal host cell is a Mucor miehei cell. In another most preferred embodiment, the filamentous fungal host cell is a Myceliophthora thermophilum cell. In another most preferred embodiment, the filamentous fungal host cell is a Neurospora crassa cell. In another most preferred embodiment, the filamentous fungal host cell is a Penicillium purpurogenum cell. In another most preferred embodiment, the filamentous fungal host cell is a Thielavia terrestris cell or an Acremonium chrysogenum cell. In another most preferred embodiment, the Trichoderma cell is a Trichoderma harzianum, Trichoderma koningii, Trichoderma longibrachiatum, Trichoderma reesei or Trichoderma viride cell. The use of Aspergillus spp. for the expression of proteins is described in, e.g., EP 272 277, EP 230 023.

The nucleotide sequences, DNA, of the invention may be modified such as to optimize the codon usage for a preferred particular host organism in which it will be expressed. Examples of this are published for yeast (Woo J H, et al, Protein Expression and Purification, Vol. 25 (2), pp. 270-282, 2002), fungi (Te'o et al, FEMS Microbiology Letters, Vol. 190 (1) pp. 13-19 (2000)), and other microorganisms, as well as for Der p 1 expressed in mammalian cells (Massaer M, et al, International Archives of Allergy and Immunology, Vol. 125 (1), pp. 32-43, 2001).

Methods of Preparing Group 2 Mite Polypeptide Variants.

The polypetide variants of the invention may be prepared by (a) transforming a suitable host cell with a nucleotide construct of the invention, (b) cultivating the recombinant host cell of the invention comprising a nucleotide construct of the invention under conditions conducive for production of the variant of the invention and (c) recovering the variant. The method may in a particular embodiment be carried out as described in Hakkaart et al. (Clin. and Exp. Allergy 1998, vol. 28, p. 45-52) describing recombinant expression of group 2 mite proteins where the Der p 2 signal sequence has been replaced with a yeast invertase signal peptide to enhance expression of Der p 2 in yeast.

Transformation

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81:1470-1474. A suitable method of transforming Fusarium species is described by Malardier et al., 1989, Gene 78:147-156 or in copending U.S. Ser. No. 08/269,449. Examples of other fungal cells are cells of filamentous fungi, e.g., Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277 and EP 230 023. The transformation of F. oxysporum may, for instance, be carried out as described by Malardier et al., 1989, Gene 78: 147-156.

Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153:163; and Hinnen et al., 1978, Proceedings of the National Academy of Sciences USA 75:1920. Mammalian cells may be transformed by direct uptake using the calcium phosphate precipitation method of Graham and Van der Eb (1978, Virology 52:546).

Transformation of insect cells and production of heterologous polypeptides therein may be performed as described in U.S. Pat. No. 4,745,051; U.S. Pat. No. 4,775,624; U.S. Pat. No. 4,879,236; U.S. Pat. No. 5,155,037; U.S. Pat. No. 5,162,222; EP 397,485) all of which are incorporated herein by reference.

Cultivation

The transformed or transfected host cells described above are cultured in a suitable nutrient medium under conditions permitting the production of the desired molecules, after which these are recovered from the cells, or the culture broth.

The medium used to culture the cells may be any conventional medium suitable for growing the host cells, such as minimal or complex media containing appropriate supplements. Suitable media are available from commercial suppliers or may be prepared according to published recipes (e.g., in catalogues of the American Type Culture Collection). The media are prepared using procedures known in the art (see, e.g., references for bacteria and yeast; Bennett, J. W. and LaSure, L., editors, More Gene Manipulations in Fungi, Academic Press, CA, 1991).

Recovery

In a particular embodiment the polypeptide variant of the invention is in an isolated and purified form. Thus the polypeptide variant of the invention is provided in a preparation which more than 20% w/w pure, particularly more than 50% w/w pure, more particularly more than 75% w/w pure, more particularly more than 90% w/w pure and even more particularly more than 95% w/w pure. The purity in this context is to be understood as the amount of polypeptide variant of the invention present in the preparation of the total polypeptide material in the preparation.

When applied to a polypeptide, the term “isolated” indicates that the polypeptide is found in a condition other than its native environment, such as apart from blood and animal tissue. In a preferred form, the isolated polypeptide is substantially free of other proteins, particularly other proteins of animal origin. It is preferred to provide the polypeptides in a highly purified form, i.e., greater than 95% pure, more preferably greater than 99% pure.

If the molecules are secreted into the nutrient medium, they can be recovered directly from the medium. If they are not secreted, they can be recovered from cell lysates. The molecules are recovered from the culture medium by conventional procedures including separating the host cells from the medium by centrifugation or filtration, precipitating the proteinaceous components of the supernatant or filtrate by means of a salt, e.g., ammonium sulphate. The proteins may be matured in vitro e.g., by acidification to induce autocatalytic processing (Jacquet et al., Clin Exp Allergy, 2002, vol. 32 pp 1048-53), and they may be purified by a variety of chromatographic procedures, e.g., ion exchange chromatography, gelfiltration chromatography, affinity chromatography, or the like, dependent on the type of molecule in question (see, e.g., Protein Purification, J-C Janson and Lars Ryden, editors, VCH Publishers, New York, 1989).

Activation of Polymers

In case the variant of the invention is to be conjugated to one or more polymers and if the polymeric molecules to be conjugated with the polypeptide in question are not active it must be activated by the use of a suitable technique. It is also contemplated according to the invention to couple the polymeric molecules to the polypeptide through a linker. Suitable linkers are well-known to the skilled person.

Methods and chemistry for activation of polymeric molecules as well as for conjugation of polypeptides are intensively described in the literature. Commonly used methods for activation of insoluble polymers include activation of functional groups with cyanogen bromide, periodate, glutaraldehyde, biepoxides, epichlorohydrin, divinylsulfone, carbodiimide, sulfonyl halides, trichlorotriazine etc. (see R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Boca Raton; G. T. Hermanson et al., (1993), “Immobilized Affinity Ligand Techniques”, Academic Press, N.Y.). Some of the methods concern activation of insoluble polymers but are also applicable to activation of soluble polymers e.g., periodate, trichlorotriazine, sulfonylhalides, divinylsulfone, carbodiimide etc. The functional groups being amino, hydroxyl, thiol, carboxyl, aldehyde or sulfydryl on the polymer and the chosen attachment group on the protein must be considered in choosing the activation and conjugation chemistry which normally consist of i) activation of polymer, ii) conjugation, and iii) blocking of residual active groups.

In the following a number of suitable polymer activation methods will be described shortly. However, it is to be understood that also other methods may be used.

Coupling polymeric molecules to the free acid groups of polypeptides may be performed with the aid of diimide and for example amino-PEG or hydrazino-PEG (Pollak et al., (1976), J. Amr. Chem. Soc., 98, 289□291) or diazoacetate/amide (Wong et al., (1992), “Chemistry of Protein Conjugation and Crosslinking”, CRC Press).

Coupling polymeric molecules to hydroxy groups are generally very difficult as it must be performed in water. Usually hydrolysis predominates over reaction with hydroxyl groups. Coupling polymeric molecules to free sulfhydryl groups can be reached with special groups like maleimido or the ortho-pyridyl disulfide. Also vinylsulfone (U.S. Pat. No. 5,414,135, (1995), Snow et al.) has a preference for sulfhydryl groups but is not as selective as the other mentioned.

Accessible Arginine Residues in the Polypeptide Chain May be Targeted by Groups Comprising Two Vicinal Carbonyl Groups.

Techniques involving coupling electrophilically activated PEGs to the amino groups of Lysines may also be useful. Many of the usual leaving groups for alcohols give rise to an amine linkage. For instance, alkyl sulfonates, such as tresylates (Nilsson et al., (1984), Methods in Enzymology vol. 104, Jacoby, W. B., Ed., Academic Press: Orlando, p. 56□66; Nilsson et al., (1987), Methods in Enzymology vol. 135; Mosbach, K., Ed.; Academic Press: Orlando, pp. 65□79; Scouten et al., (1987), Methods in Enzymology vol. 135, Mosbach, K., Ed., Academic Press: Orlando, 1987; pp 79□84; Crossland et al., (1971), J. Amr. Chem. Soc. 1971, 93, pp. 4217_{—}4219), mesylates (Harris, (1985), supra; Harris et al., (1984), J. Polym. Sci. Polym. Chem. Ed. 22, pp 341□352), aryl sulfonates like tosylates, and para-nitrobenzene sulfonates can be used.

Organic sulfonyl chlorides, e.g., Tresyl chloride, effectively converts hydroxy groups in a number of polymers, e.g., PEG, into good leaving groups (sulfonates) that, when reacted with nucleophiles like amino groups in polypeptides allow stable linkages to be formed between polymer and polypeptide. In addition to high conjugation yields, the reaction conditions are in general mild (neutral or slightly alkaline pH, to avoid denaturation and little or no disruption of activity), and satisfy the non-destructive requirements to the polypeptide.

Tosylate is more reactive than the mesylate but also more unstable decomposing into PEG, dioxane, and sulfonic acid (Zalipsky, (1995), Bioconjugate Chem., 6, 150□165). Epoxides may also been used for creating amine bonds but are much less reactive than the above mentioned groups.

Converting PEG into a chloroformate with phosgene gives rise to carbamate linkages to Lysines. This theme can be played in many variants substituting the chlorine with N-hydroxy succinimide (U.S. Pat. No. 5,122,614, (1992); Zalipsky et al., (1992), Biotechnol. Appl. Biochem., 15, p. 100□114; Monfardini et al., (1995), Bioconjugate Chem., 6, 62□69, with imidazole (Allen et al., (1991), Carbohydr. Res., 213, pp 309□319), with para-nitrophenol, DMAP (EP 632 082 A1, (1993), Looze, Y.) etc. The derivatives are usually made by reacting the chloroformate with the desired leaving group. All these groups give rise to carbamate linkages to the peptide.

Furthermore, isocyanates and isothiocyanates may be employed yielding ureas and thioureas, respectively.

Amides may be obtained from PEG acids using the same leaving groups as mentioned above and cyclic imid thrones (U.S. Pat. No. 5,349,001, (1994), Greenwald et al.). The reactivity of these compounds is very high but may make the hydrolysis to fast.

PEG succinate made from reaction with succinic anhydride can also be used. The hereby comprised ester group make the conjugate much more susceptible to hydrolysis (U.S. Pat. No. 5,122,614, (1992), Zalipsky). This group may be activated with N-hydroxy succinimide.

Furthermore, a special linker can be introduced. The oldest being cyanuric chloride (Abuchowski et al., (1977), J. Biol. Chem., 252, 3578_{—}3581; U.S. Pat. No. 4,179,337 (1979), Davis et al.; Shafer et al., (1986), J. Polym. Sci. Polym. Chem. Ed., 24, 375-378.

Coupling of PEG to an aromatic amine followed by diazotation yields a very reactive diazonium salt which in situ can be reacted with a peptide. An amide linkage may also be obtained by reacting an azlactone derivative of PEG (U.S. Pat. No. 5,321,095, (1994), Greenwald, R. B.) thus introducing an additional amide linkage.

As some polypeptides do not comprise many Lysines it may be advantageous to attach more than one PEG to the same Lysine. This can be done e.g., by the use of 1,3-diamino-2-propanol.

PEGs may also be attached to the amino-groups of the polypeptide with carbamate linkages (WO 95/11924, Greenwald et al.). Lysine residues may also be used as the backbone.

The coupling technique used in the examples is the N-succinimidyl carbonate conjugaion technique descried in WO 90/13590 (Enzon).

Compositions

The present invention also relates to a composition comprising a variant of the invention and optionally a pharmaceutically acceptable carrier and/or adjuvant and a method for preparing such a composition comprising admixing the variant of the invention with an acceptable pharmaceutical carrier and/or adjuvant. In a particular embodiment the composition is a vaccine suitable for treating an immunological disorder, such as allergy in animals or humans.

Further the present invention relates to a composition comprising a variant of the invention in combination with one or more allergens or modified allergens, where said allergens in particular may originate from Dermatophagoides pteronyssinus, Dermatophagoides farinae, Dermatophagoides siboney, Dermatophagoides microceaus, Blomia tropicalis and Euroglyphus maynei, and said modified allergens originate from the introduction of one or more mutations in allergens originating from Dermatophagoides pteronyssinus, Dermatophagoides farinae, Dermatophagoides siboney, Dermatophagoides microceaus, Blomia tropicalis and Euroglyphus maynei. Further the present invention also relates to a composition comprising the afore mentioned allergens or modified allergens in combination with a pharmaceutically acceptable carrier and/or adjuvant and a method for preparing such a composition comprising admixing the afore mentioned allergens or modified allergens with an acceptable pharmaceutical carrier and/or adjuvant. In a particular embodiment the composition is suitable for treating an immunological disorder, such as allergy in animals or humans, such as a vaccine.

Non-limiting examples of pharmaceutical carriers and/or adjuvants includes saline, glycerol, aluminium hydroxide, aluminium phosphate, calcium phosphate, saponins (e.g., Q21 and Quill A), squalene based emulsions (e.g., MF59), monophosphoryl lipid A (and synthetic mimics), polylactide co-glycolid (PLG) particles, ISCOMS, liposomes, chitosan, bacterial DNA (e.g., unmethylated CpG containing sequences). Suitable carriers also include pharmaceutically acceptable solvents and/or tabletting aids/auxilliaries.

Use of Group 2 Mite Polypeptide Variants and Compositions Containing them

In a further aspect the invention provide use of the variant or the composition of the invention as a medicament, particularly for the treatment of an immunological disorder, such as allergy in animals and humans and/or for the preparation of a medicament for the treatment of such immunological disorder.

Traditionally, allergy vaccination is performed by parenteral, intranasal, or sublingual administration in increasing doses over a fairly long period of time, and results in, so called, desensitisation of the patient. The exact immunological mechanism is not known, but induced differences in the phenotype of allergen specific T and B cells are thought to be of particular importance.

Compared to conventional types of vaccination, allergy vaccination is complicated by the existence of an ongoing immune response in allergic patients. This immune response is characterised by the presence of allergen specific IgE, that will mediate the release of allergic mediators, thereby inducing allergic symptoms upon exposure to allergens. Thus, allergy vaccination using native and/or naturally occurring allergens has an inherent risk of side effects being in the utmost consequence life threatening to the patient.

Approaches to circumvent this problem may be divided in three categories. In practise measures from more than one category are often combined. First category of measures includes the administration of several small and increasing doses over a long period to reach a substantial aqccumulated dose. The theory being, that the protective immune response slowly is allowed to be initiated, before potentially anaphylactic doses of allergen is administrated. Second category of measures includes physical modification of the allergen by incorporation of the allergen into e.g., a gel formulation such as a aluminium hydroxide. Aluminium hydroxide has an adjuvant effect and a depot effect of slow allergen release, thus reducing the the tissue concentration of the allergen. Third category of measures include as described herein modification of the alergen for the purpose of reducing allergenicity.

The immunotherapeutic effect of an allergy vaccine can be assessed in a number of different ways. One is to measure the specific IgE binding, the reduction of which indicates a better safety profile (however not necessarily a better vaccine potential) (WO 99/47680, ALK-ABELLÓ). Also, several cellular assays could be employed to show the modified immuneresponse indicative of good allergy vaccine potential as shown in several publications, all of which are hereby incorporated by reference (van Neerven et al., “T lymphocyte responses to allergens: Epitope-specificity and clinical relevance”, Immunol Today, 1996, vol. 17, pp. 526-532; Hoffmann et al., Allergy, 1999, vol. 54, pp. 446-454, WO99/07880). Basophil histamine release: e.g., Swoboda et al., Eur. J. Immunol., vol. 32, pp 270-280, 2002.

Also skin prick testing could be employed for example as described in Kronqvist et al Clin Exp Allergy 2000 vol 30 pp 670-676

Eventually, clinical trials with allergic patients could be employed using cellular or clinical end-point measurements. (Ebner et al., Clin. Exp. All., 1997, vol. 27, pp. 107-1015; Int. Arch. Allergy Immunol., 1999, vol. 119, pp 1-5).

### EXAMPLES

### Example 1

### Finding of Epitope Patterns Within Oligo Peptides with Antibody Reactivity

A high diversity library of phages expressing random oligomeric peptides (hexa, hepta, octa, nona and/or dodeca peptides) as part of their surface proteins, were screened for their capacity to bind antibodies. The phage libraries were obtained from Schafer-N, Copenhagen, Denmark.

Antibody samples were raised in animals (Rat, Rabbits or Mice) by parenteral or mucosal administration of each of the proteins listed below. The antibodies were dissolved in phosphate buffered saline (PBS). In some cases, antibodies of specific subclasses were purified from the serum of immunised animals by capryilic acid precipitation (for total IgG) or by affinity chromatography using paramagnetic immunobeads (Dynal AS) loaded with one of the following antibodies: mouse anti-rat IgG1 or rat anti-mouse IgE.
1) amylase AA560 from Bacillus sp. DSM 12649, (Rat IgG)
2) alpha-amylase of Bacillus halmapalus (WO96/23873), which is also called amylase SP722 (Rat IgG)
3) a variant of SP722 with residues 183 and 184 deleted, called JE-1 (WO96/23873), (Rat IgG and Rabbit IgG)
4) Mycelioptora thermopila laccase (WO 95/33836) (Rabbit IgG),
5) T. lanuginosus lipase (Lipolase™) (Rat IgG and Rabbit IgG),
6) family 45 cellulase from Humicola insolens (Carezyme™) (Rabbit IgG),
7) Bacillus lentus protease (Savinase™) (Rat IgG, Mouse IgG, Mouse IgE, and Rabbit IgG),
8) Subtilisin Novo (BPN') from B. amyloliquefaciens (Rat IgG),
9) The Y217L variant of Subtilisin Novo (Rat IgG),
10) Subtilisin Carlsberg (Alcalase™) (Rat IgG),
11) TY145 protease (Rat IgG),
12) CDJ31 protease,
13) Subtilisin 147 (Esperase™) (Rat IgG),
14) Bacillolysin from Bacillus amyloliquefaciens (Neutrase™) (Rat IgG and Rat IgG1), and
15) Subtilisin PD498 (WO 93/24623) (Rat IgG and Rabbit IgG),
16) Der p 1 (Human IgG and IgE),
17) Ara h 2 (Human IgG and IgE),
18) Gly m 4 (Human IgG and IgE),
19) Pru v 1 (Human IgG and IgE),
20) Ara h 8 (Human IgG and IgE),
21) Betv 1 (Human IgG and IgE).

The phage libraries were incubated with the antibody coated beads. E.g. phages expressing oligo-peptides with affinity for mouse IgE antibodies were captured onto rat anti-mouse IgE-coated beads and collected by exposing these paramagnetic beads to a magnetic field. The collected phages were eluted from the immobilised antibodies by mild acid treatment, or by elution with itact protein antigen specific for the respective antibody sample (e.g., Savinase for anti-Savinase antibodies). The isolated phages were amplified using methods known in the art. Alternatively, immobilised phages were directly incubated with E. coli for infection. In short, F-factor positive E. coli (e.g., XL-1 Blue, JM101, TG1) were infected with M13-derived vector in the presence of a helper phage (e.g., M13K07), and incubated, typically in 2xYT containing glucose or IPTG, and appropriate antibiotics for selection. Finally, cells were removed by centrifugation. This cycle of events was repeated on the respective cell supernatants, minimum 2 times and maximum 5 times. After selection round 2, 3, 4 and 5, a fraction of the infected E. coli was incubated on selective 2xYT agar plates, and the specificity of the emerging phages was assessed immunologically: Phages were transferred to a nitrocellulase (NC) membrane. For each plate, 2 NC-replicas were made. One replica was incubated with the selection antibodies, the other replica was incubated with the selection antibodies and the immunogen used to obtain the antibodies as competitor. Those plaques that were absent in the presence of immunogen, were considered specific, and were am-plified according to the procedure described above.

The specific phage-clones were isolated from the cell supernatant by centrifugation in the presence of polyethylenglycol. DNA was isolated, the DNA sequence coding for the oligopeptide was amplified by PCR, and its DNA sequence was determined, all according to standard procedures known in the art. The amino acid sequence of the corresponding oligopeptide was deduced from the DNA sequence.

These 1060 experimentally determined reactive peptides were supplemented with information on 420 reactive peptides published in the literature:
Allergy 38 (1983) 449-459,
Allergy 56 (2001) 118-125;
Allergy 56 s67 (2001) 48-51;
Allergy 54 (1999) 1048-1057;
Arch Biochem Biophys 342 (1997) 244-253
B. B. Res. Com. 219 (1996) 290-293;
Biochem J 293 (1993) 625-632;
Bioinformatics 18 (2002) 1358-1364;
Clin Exp Allergy 24 (1994) 100-108;
Clin Exp Allergy 24 (1994) 250-256;
Clin Exp Allergy 31 (2001) 331-341;
Clin Exp Med 24 (1994) 100-108;
Eur J Biochem 245 (1997) 334-339;
Int Arch Allergy Appl Immunol 89 (1989) 342-348
Int Arch Allergy Appl Immunol 89 (1989) 410-415
Int Arch Appl Immunol 103 (1994) 357-364
Int Arch Appl Immunol 92 (1990) 30-38
J Allergy Clin Immunol 106 (2000) 150-158
J Allergy Clin Immunol 107 (2001) 1069-1076
J Allergy Clin Immunol 93 (1994) 34-43
J Biol Chem 271 (1996) 29915-29921
J Clin Invest 103 (1999) 535-542
J Immunol 121 (1989) 275-280
J Immunol 133 (1984) 2668-2673
J Immunol 140 (1988) 611-616
J Immunol 147 (1991) 205-211
J Immunol 151 (1993) 5354-5363
J Immunol 151 (1993) 7206-7213
J Immunol Methods 213 (1998) 1-17
Mol Immunol 25 (1988) 355-365
Mol Immunol 28 (1991) 1225-1232
Mol Immunol 29 (1992) 1383-1389
Mol Immunol 29 (1992) 257-262
Mol Immunol 30 (1993) 183-189
Mol Immunol 35 (1998) 293-305
Mol Immunol 37 (2000) 789-798
Peptides 21 (2000) 589-599
Protein Scieince 8 (1999) 760-770
Scand J Immunol 27 (1988) 587-591
Science 233 (1986) 747-753
WO 90/11293
WO 99/38978
WO 01/34186
WO 01/39799
WO 01/39799
WO 01/49834
   www.csl.gov.uk/allergen

Thus, in total 1480 peptide sequences (576 IgG binding and 904 IgE binding) with specificity for the protein-specific antibodies, described above, were obtained. These sequences were collected in a database, and analysed by sequence alignment to identify epitope patterns observing that conservative alternatives were considered equal (as described above).

Identifying Epitope Patterns

In principle, each of the IgG and IgE reactive (oligo)peptide sequences represented an epitope pattern. However, in the reactive (oligo)peptide sequences some epitope patterns were redundant and to remove redundency among the epitope patterns, the reactive (oligo)peptides sequences were subjected to computerised data analysis.

First all possible dipeptides were generated corresponding to 13² different combinations taking conservative alternatives into account. The presence and frequency of each dipeptide among the 576 IgG and 904 IgE reactive (oligo)peptide sequences were listed. Next all possible tripeptides were generated coresponding to 133 different combinations and again the presence and frequency of each tripeptide among the reactive (oligo)peptide sequences were listed. All possible combinations of the listed dipeptides and tripeptides were then generated including those containing 1, 2, 3 or 4 residues inserted bewteen the dipeptides and tripeptides, these residues selected among the 13 possible residue types. This procedure generated a list of different peptide combinations of 5 to 9 amino acids each containing at least one dipeptide and at lest one tripeptide from the initial listings as well as 0 to 4 residues in between. The frequency of each peptide combination among the reactive (oligo)peptide sequences were then ranked and relevant epitope patterns were selected by a procedure where reactive peptides covered by the most frequent combination were first selected and separated from the group of the reactive peptides. Then reactive peptides covered by the second most frequent combination were selected and separated from the remaining group. Then reactive peptides covered by the third most frequent combination were selected and separated from the remaining group. This procedure was repeated until combinations covering all reactive peptides are found. This way it was found that 403 (IgG) and 592 (IgE) combinations (epitope patterns) were found to cover all the 576 IgG and 904 IgE reactive peptides.

### Example 2

### Predicting Epitopes

The Der p 2 (pdb #; 1KTJ, 1A9V) and Der f 2 (1AHK, 1AHM) models were taken from the pdb database.

Surface accesibility was measured for each amino amino acid in SEQ ID NO:1 using the DSSP program (see W. Kabsch and C. Sander, Biopolymers 22 (1983) 2577-2637) in percent of a standard value for that amino acid. The standard values generated according to established methods by analysing average surface accesibility of an amino acid in a 20-mer homogeneous peptide in helix formation using the DSSP program. For each of the 13 different residue types (taking consertive alternatives into consideration) the average surface accesibility were as follows:

| Residue | Accessiblity Å² |
|---|---|
| A | 62 |
| C | 92 |
| D | 69 |
| E | 156 |
| F | 123 |
| G | 50 |
| H | 130 |
| I | 84 |
| K | 174 |
| L | 97 |
| M | 103 |
| N | 85 |
| P | 67 |
| Q | 127 |
| R | 211 |
| S | 64 |
| T | 80 |
| V | 81 |
| W | 126 |
| Y | 104 |

Epitopes were predicted by a computer program on a 3-dimensional model of Der p 2 (SEQ ID NO:1) and Der f 2 (SEQ ID NO:2) by using the epitope patterns found in example 1 as follows:
(1) For all amino acids it was examined if (a) the amino acid type match the first amino acid of an epitope patterns and (b) the solvent surface accessibility greater than or equal to a predefined value, e.g. 50%. Those amino acid satisfying 1(a) and 1(b) are selected.
(2) For all amino acids within a distance of 10 Å from the amino acids selected in step 1 it is examined if (a) the amino acid type matches the second amino acid of the pattern and (b) the surface accessibility greater than or equal to the predefined value, e.g., 50%. Those amino acid satisfying 2(a) and 2(b) are selected
(3) For all amino acids within a distance 10 Å from the amino acids selected in step 2 it is examined if (a) the amino acid type matches the third amino acid of the pattern and (b) the surface accessibility greater than or equal to the predefined value, e.g. 50%. Those amino acid satisfying 3(a) and 3(b) are selected.
(4) Repeating step 3 for all amino acids in the epitope pattern

Further, a limit of 25 Å was set as the maximum distance between any two epitope residues.

This procedure was carried out for all epitope patterns for each of the following settings for surface accessibility cutoff: 40%, 60% and 70% for Der p 2, and cutoff: 50% for Der f 2. Epitope patterns finding a match on the 3 dimensional structure of SEQ ID NO: 1, respectively SEQ ID NO: 2, according this procedure are predicted as epitopes.

### Example 3

### Construction, Expression and Purification of Variants

Der p 2 variants of the invention comprising specific substitutions can be made by cloning of DNA fragments (Sambrook et al., Molecular Cloning: A Laboratory Manual, 2nd Ed., Cold Spring Harbor, 1989) produced by PCR with oligos containing the desired mutations. The template plasmid DNA may be pSteD212, or an analogue of this containing Der p 2 or a variant of Der p 2. Mutations are introduced by oligo directed mutagenesis to the construction of variants. The Der p 2 plasmid constructs are transformed into S. cerevisiae, strain JG169, as described by Becker and Guarente (1991, Methods Enzymology, 194: 182-187).

The group 2 allergen or variants hereof of the present invention are located in vector pSteD212, which is derived from yeast expression vector pYES 2.0 (Invitrogen, Okkels, Ann. New York Acad. Sci. 1996, vol 728 p. 202-207).

This plasmid replicated both in E. coli and in S. cerevisiae. In S. cerevisiae Der p 2 or variants hereof according to the invention are expressed from this plasmid. The Der p 2 variants are confirmed by DNA sequencing.

Fermentation

Fermentations for the production of Der p 2 protein or variants hereof are performed at 30 degree C. on a rotary shaking table (250 r.p.m.) in 500 ml baffled Erlenmeyer flasks containing 100 mL SC medium for 5 days.

Consequently, in order to make e.g. a 2 litre broth 20 Erlenmeyer flasks are fermented simultaneously.

SC Medium (per litre):

| | | |
|---|---|---|
| Yeast Nitrogen Base without amino acids | 7.5 | g |
| Succinic acid | 11.3 | g |
| Casamino acid without vitamine | 5.6 | g |
| Tryptophan | 0.1 | g |

Add H₂O. Autoclave and cool before adding glucose and L-threonin to a final concentration of 4% and 0.02%, respectively.

For agar plates, 20 g bactoagar is added to the medium before autoclave.

Method for Semi Purification of Der p2.

1 L fermentation supernatant of Der p2 antigen (Dermatophagoides pteronyssinus) expressed in Yeast was centrifuged and precipitate containing cell debris was discarded. The cell supernatants were then sterile filtered under pressure through 0.22 micro m sterile filter PES (Polyethersulfone) obtained from Corning (One Riverfront Plaza, Corning, N.Y. 14831, USA).

Hydrophobic interaction chromatography was carried out on 50 mL XK26 column purchased from Amersham-Pharmacia which was packed with Phenyl Sepharose™ 6 Fast Flow (Amersham-Bioscience).

The column was washed then equilibrated with 1.5 M ammonium sulphate dissolved in 25 mM Sodium Acetate pH 5.

The sterile filtrated fermentation supernatant was applied on the column with a flow of 10 mL per minute. Unbound material was then washed out using 1.5 M ammonium sulphate dissolved in 25 mM sodium Acetate buffer pH 5 (Buffer B). When all the unbound material was washed out from the column, which was detected by absorbance reading at 280 nm, Der p2 was then eluted with buffer A containing 25 mM Sodium Acetate pH 5 without any other salt. 10 mL fractions were collected. Fractions were tested on ELISA as described in example 5, and absorbance measured at 280 nm to determine when Der p2 was eluted. Fractions containing the desired protein were checked by SDS-PAGE followed by total staining with Coomasie Blue. Fractions containing Protein with molecular weights around 14 kDa and found immunoreactive in the qualitative tests described above were then pooled. Further purification can be done using ion-exchange chromatography or gel filtration.

### Example 4

### In Vivo Assessment of Allergenicity of an Enzyme Variant (MINT Assay)

Mouse intranasal (MINT) model (Robinson et al., Fund. Appl. Toxicol. Vol. 34, pp. 15-24, 1996) can be used to verify allergenicity of group 2 mite polypeptide variants.

Mice are dosed intranasally with the group 2 mite polypeptide variant on the first and third day of the experiment and from thereon on a weekly basis for a period of 6 weeks. Blood samples are taken 15, 31 and 45 days after the start of the study, and the serum can subsequently be analysed for IgE levels.

Measurement of the Concentration of Specific IgE in Mouse Serum by ELISA:

The relative concentrations of specific IgE antibodies in serum samples from mice are measured by a three layer sandwich ELISA according to the following procedure:
1) The ELISA-plate (Nunc Maxisorp) is coated with 100 microliter/well rat anti-mouse IgE Heavy chain (HD-212-85-IgE3 diluted 1:100 in 0.05 M Carbonate buffer pH 9.6). Incubated over night at 4° C.
2) The wells are emptied and blocked with 200 microliter/well 2% skim milk in 0.15 M PBS buffer pH 7.5 for 1 hour at 4° C. The plates are washed as before.
3) The plates are incubated with dilutions of mouse sera (100 microL/well), starting from an 8-fold dilution and 2-fold dilutions hereof in 0.15 M PBS buffer with 0.5% skim milk and 0.05% Tween20. Appropriate dilutions of positive and negative control serum samples plus buffer controls are included. Incubated for 1 hour at room temperature. Gently shaken. The plates are washed 3 times in 0.15 M PBS buffer with 0.05% Tween20.
4) 100 microliter/well of group 2 mite polypeptide variant diluted to 1 microgram protein/ml in 0.15 M PBS buffer with 0.5% skim milk and 0.05% Tween20 is added to the plates. The plates are incubated for 1 hour at 4° C. The plates are washed as before.
5) Specific polyclonal anti-group 2 mite polypeptide variant antiserum serum (pig) for detecting bound antigen is diluted in 0.15 M PBS buffer with 0.15% skim milk and 0.05% Tween20. 100 microl/well and incubated for 1 hour at 4° C. The plates are washed as before.
6) 100 microliter/well pig anti-rabbit Ig conjugated with peroxidase diluted 1:1000 in 0.15 M PBS buffer with 0.5% skim milk and 0.05% Tween20 is added to the plates. Incubated for 1 hour at 4° C. The plates are washed as before.
7) 100 μl/well TMB (Plus (Ready-to-go substrate; Kem-En-Tec, Cat. No.: 4390A) is added, and the reaction is allowed to run for 10 min.
8) The reaction is stopped by adding 100 microliter/well 1M H₂SO₄.
9) The plates are read at 450 nm with 620 nm as reference.

The dose response curves are graphed, and fitted to a sigmoid curve using non-linear regression, and the EC50 is calculated for the group 2 mite polypeptide variant.

Measurement of the Concentration of Specific IgG1 in Mouse Serum by ELISA

The relative concentrations of specific IgG1 antibodies in serum samples from mice are measured by a three layer sandwich ELISA according to the following procedure:
1) The ELISA-plate (Nunc Maxisorp) is coated with 100 microliter/well of group 2 mite polypeptide variant diluted in PBS to 10 microg/ml. Incubated over night at 4° C.
2) The wells are emptied and blocked with 200 microliter/well 2% skim milk in 0.15 M PBS buffer pH 7.5 for 1 hour at 4° C. The plates are washed 3 times with 0.15 M PBS buffer with 0.05% Tween20.
3) The plates are incubated with dilutions of mouse sera (100 microL/well), starting from an 20-fold dilution and 3-fold dilutions hereof in 0.15 M PBS buffer with 0.5% skim milk and 0.05% Tween20. Appropriate dilutions of positive and negative control serum samples plus buffer controls are included. Incubated for 1 hour at room temperature. Gently shaken. The plates were washed as before.
4) 100 microliter/well biotinylated Rat-anti-mouse IgG1 (Serotec, Cat. No.: MCA 336B), diluted 2000× in 0.15 M PBS buffer with 0.5% skim milk and 0.05% Tween20 is added to the plates. The plates are incubated for 1 hour at 4° C. The plates are washed as before.
5) 100 microliter/well Horseradish Peroxidase-conjugated Streptavidin (Kierkegaard & Perry, Cat. No.: 14-30-00), diluted 1000× in 0.15 M PBS buffer with 0.15% skim milk and 0.05% Tween20. Incubated for 1 hour at 4° C. The plates are washed as before.
6) 100 microliter/well TMB (Plus (Ready-to-go substrate; Kem-En-Tec, Cat. No.: 4390A) is added, and the reaction is allowed to run for 10 min.
7) The reaction is stopped by adding 100 microliter/well 1M H₂SO₄.
8) The plates are read at 450 nm with 620 nm as reference.

The dose response curves are plotted and fitted to a sigmoid curve using non-linear regression, and the EC50 is calculated for the group 2 mite polypeptide variant.

### Example 5

### In Vitro Assessment of IgE-Antigenicity of an GROUP 2 Mite Allergens

Reduced IgE binding is verified in vitro by direct or competitive ELISA (or similar solid phase immunochemical assays) or Basophil histamine release. Group 2 mite polypeptide variants with reduced IgE-antigenicity can then be tested further in vivo, by skin prick testing.

Direct ELISA

Immunoplates (Nunc Maxisorb; Nunc-Nalgene) are coated overnight at 4° C. with a suitable dose, or dose-range, of natural or recombinant group 2 mite polypeptide variant allergen, or variants thereof. The plates are then washed thoroughly with Phosphate Buffered Saline (PBS) containing 0.05% Tween 20 (PBST), and remaining binding sites are blocked with PBS containing 1% Skim Milk Powder (SMP). Sera from patients allergic to dust mites, with a positive RAST value, is diluted 1/8 in PBST and added to the plates and incubated at 4° C. for a suitable time period. Following a thorough wash with PBST, the allergen-IgE complexes are detected, by serial incubation with an rabbit anti-human IgE antibody (DAKO), and goat anti-rabbit Ig coupled to horseradish peroxidase. The enzymatic activity is measured by adding “TMB plus” substrate (Kem-En-Tec), and stopping the reaction with an equal volume of 0.2 M H₂SO₄, and quantitaing colour development by measuring optical density at 450 nm (OD450) in an ELISA plate reader. OD450 will reflect IgE binding to the allergen, and it is thus possible to compare binding to the variants to that of the wild type polypeptide.

Competitive ELISA

Is carried out like direct ELISA, with two exeptions: the Immunoplates are coated with a fixed concentration of wild type polypeptide, and the diluted serum from allergic patients is preincubated with a dose range of recombinant wild type or variant allergen. If the IgE binds to the polypeptide in solution, it will reduce binding to the platebound wild type polypeptide, thus reducing the OD450.

Basophil Histamine Release

The basophil containing cell fraction is isolated from whole blood from donors allergic to group 2 mite polypeptides, by centrifugation. The cells are then incubated with a dose range of recombinant group 2 mite polypeptide variant allergen. IgE binding will crosslink IgE on the surface of the basophile granulocytes, thereby releasing histamine into the surroundings. Liberated histamine can then by measured by, e.g., fluorumetric methods (see e.g., Nolte et al., Allergy, vol. 42, pp. 366-373, 1987).

Skin Prick Testing

Is carried out on patients allergic to house dust mites, and the technique is well known in the art, e.g., Kronquist et al., Clin. Exp. Allergy, 2000, vol. 30, pp. 670-676).

Briefly, 15 micro L of solutions containing the recombinant wild type or variant allergens is placed on the patient's forearm. Thereafter, the skin is pricked with a Prick-Lancette (ALK). The test sites are placed 3 cm apart to avoid false-positive results. From an initial solution of recombinant allergens (e.g., 1 mg protein/mL), suitable serial dilutions (e.g., from 100 micro g to 0.1 micro g/mL) are made in sterile physiologic saline solution. These dilutions are selected according to the concentration allergens, which elicited significant histamine release by sensitized basophils. It has been shown that the thresholds of positivity for histamine release tests and intradermal reactions are in the same range; and it is assumed that the sensitivity of prick tests is 10² to 10³ times lower than that of intradermal tests. A negative control test is performed with saline solution, and a positive control test is done with histamine at e.g., 1 mg/mL. The diameter of the weal is used as a measure of allergenic reactivity towards that variant, and this allows for comparison of the variant allergens to the parent or native type allergen.

### Example 6

### Assessing Retained Ability to Stimulate T Cells

The lymphocyte fraction from heparinized blood from patients allergic to group 2 mite polypeptides is purified, e.g., by density gradient centrifugation on Lymphoprep (Axis-Shield PoC, Norway), and resuspended in a suitable growth medium, e.g., RPMI 1640 supplemted with 10% human AB serum and L-glutamine, and plated at a suitable density (e.g., 200,000 cells/well) in a 96 well sterile tissue culture plate (e.g., Nunclon Delta). Suitable Serial dilutions (e.g., from 200-0.2 microg/ml) of group 2 mite polypeptide variant allergens are made up in growth media and added to the cells, together with a media-only control. The plates are then incubated for 7 days at 37° C., 5% CO₂, 100% Humidity. At the end of the incubation, T cell proliferation is measured by a suitable conventional method, such as, incorporation of ³H Thymidine, MTT reduction or AlamarBlue assay (Serotec). The group 2 mite polypeptide variant and the parent group 2 mite polypeptide allergen should have similar dose-response profiles.

### Example 7

### Epitope Mapping Based on Human Anti-Der p 2 Antiserum

Preparation of Human IgE Beads with Specificity for Der p 2 for Targeting in Selection Experiments (Anti-Der p 2 Beads):

Rabbit anti-human IgE (anti-hIgE) antibodies were covalently linked to commercially available tosyl-activated paramagnetic beads. After inactivation of the remaining linkage sites and washing of the beads according to manufacturer's specification, the anti-hIgE-beads were incubated overnight at 4° C. with pooled sera from patients sensitized to Der p 2 (the sera were 3× diluted in dilution buffer (PBS at pH 6.6)). 200 microlitre beads were washed 3× with 40 ml of washing buffer (PBS at pH6.6 plus 0.05% Tween20), then incubated with PBS supplemented with 2% skim milk for an hour at room temperature and washed 3× as before.

Selection of epitope mimics from commercially available phage-displayed peptide libraries: Epitope mimicking peptides were isolated from commercially available phage display libraries of either 7mer, constrained 7mer or 12mer peptide libraries (New England Biolabs). The anti-Der p 2-beads were incubated with 2*10¹¹ library phages for 4 h at room temperature, after which unbound phages were removed by extensive washing. To avoid the enrichment of peptides that were bound to either plain beads or to the anti-hIgE antibody, a specific elution procedure was implemented: After washing, beads were first incubated with PBS supplemented with 0.5% skim milk for an hour at room temperature. After this additional washing step, phages were eluted from the beads by incubation with 25 microM purified Der p 2 in PBS supplemented with 0.5% skim milk, again lasting an hour at room temperature. Only phages in the supernatant of this elution were propagated further. Selected phages were amplified according to the guidelines of the library manufacturer (NEB user manual) after a first round of selection using ER2738 cells. After a second round, cells were infected and spread out for isolation of phages, which were subsequently tested for binding and sequenced.

Phage ELISA to Test Binding to Serum IgE:

100 ng of anti-hIgE antibody in 100 microlitre coating buffer (50 mM sodiumcarbonate at pH 9.0) were coated overnight at 4° C. on 96-well plates. Unoccupied binding sites were blocked for 2 hrs at room temperature with skimmed milk (2 (wt/vol) % in washing buffer (PBS at pH6.6 plus 0.05% Tween20). Human IgE are then selectively immobilized from serum samples that were diluted 50× in dilution buffer (PBS at pH 6.6) to 100 microlitre and incubated for 2 hr at room temperature. Phage preparations from isolated cells infected with 2^{nd} round elution, which contained phages displaying putative epitope mimics, were sampled in varying dilutions for their binding. Bound phages were detected either with mouse anti M13-phage antibody-horseradish peroxidase (HRP) conjugates or with a mouse anti-pill Antibody, Sheep anti-mouse IgG antibody-HRP sandwich when cross-reactivity to the serum in absence of any phage preparation was detected.

### Example 8

### Screening for Der p 2 Variants

For screening of yeast transformants expressing Der p 2 or Der p 2 variants, the transformation solution is plated on SC-agar plates for colony formation at 30 degree C., 3 days. Colonies is inoculated in 96 micro-well plates, each well containing 200 microL SC medium. The plates are fermented at 30 degree C., 250 r.p.m. for 5 days.

50 microL culture broth is diluted 1:1 in 0.15 M Phosphate Buffered Saline (PBS) before OD450 measurement in sandwich ELISA. Culture broth of yeast transformed with a plasmid without the Der p 2 gene is used as background with an average OD450 of 0.55. Der p 2 variants tested in sandwich ELISA with OD450>0.55 are DNA sequenced. A part of the variants are concentration determined directly in culture broth by the sandwich ELISA technique with natural Der p 2 as a standard.

Der p 2 variants identified and determined by both OD450>0.55 and in a quantitative sandwich ELISA (concentrations given as microg/mL) are protein purified for further analysis.

1-20. (canceled)

21. A variant of a parent Der p 2 polypeptide, wherein the variant comprises one or more mutations at positions 64, 40, 53, 57, 82, 83, and 97, wherein each position corresponds to the position of the amino acid sequence of SEQ ID NO: 1.

22. The variant of claim 21, wherein the parent Der p 2 polypeptide has an animo acid sequence which is at least 80% identical to SEQ ID NO: 1.

23-25. (canceled)

26. The variant of claim 21, wherein the parent Der p 2 polypeptide has an animo acid sequence of SEQ ID NO: 1.

27. The variant of claim 21, wherein the variant has an amino acid sequence which is at least 80% identical to the amino acid sequence of the parent Der p 2 polypeptide.

28-30. (canceled)

31. The variant of claim 21, which comprises a mutation at position 64.

32-37. (canceled)

38. The variant of claim 21, wherein the one or more mutations are substitutions.

39. The variant of claim 21, which has an altered IgE-antigenicity as compared to the parent Der p 2 polypeptide.

40. The variant of claim 21, which has an altered IgG-antigenicity as compared to the parent Der p 2 polypeptide.

41. The variant of claim 21, which induces an altered immunogenic response in exposed animals as compared to the parent Der p 2 polypeptide.

42. The variant of claim 21, wherein the one or more mutations are independently substitutions of a hydrophilic amino acid to a hydrophobic amino acid, a polar amino acid to a non-polar amino acid, or an acidic amino acid to a basic amino acid.

43. The variant of claim 21, wherein the one or more mutations are insertions of one or more attachment groups for conjugating a polymer.

44. The variant of claim 21, wherein the one or more mutations are insertions of one or more glycosylation sites.

45. The variant of claim 21, which has at least the same T-cell stimulatory effect compared to the parent Der p 2 polypeptide.

46. A pharmaceutical composition comprising a variant of claim 21 and a pharmaceutically acceptable carrier or an adjuvant.

47. A nucleotide sequence encoding the variant of claim 21.

48. A nucleotide construct comprising the nucleotide sequence of claim 47, operably linked to one or more control sequences that direct the production of the variant in a host cell.

49. A recombinant expression vector comprising the nucleotide construct of claim 48.

50. A recombinant host cell comprising the nucleotide construct of claim 49.

51. A method of preparing a variant, comprising:
(a) cultivating the recombinant host cell of claim 50 under conditions conducive for production of the variant, and
(b) recovering the variant.

52. A variant of a parent Der f 2 polypeptide, wherein the variant comprises one or more mutations at positions 32, 59, 61, 62, and 98, wherein each position corresponds to the position of the amino acid sequence of SEQ ID NO: 2.

53. The variant of claim 52, wherein the parent Der f 2 polypeptide has an animo acid sequence which is at least 80% identical to SEQ ID NO: 2.

54-56. (canceled)

57. The variant of claim 52, wherein the parent Der f 2 polypeptide has an animo acid sequence of SEQ ID NO: 2.

58. The variant of claim 52, wherein the variant has an amino acid sequence which is at least 80% identical to the amino acid sequence of the parent Der f 2 polypeptide.

59-66. (canceled)

67. The variant of claim 52, wherein the one or more mutations are substitutions.

68. The variant of claim 52, which has an altered IgE-antigenicity as compared to the parent Der f 2 polypeptide.

69. The variant of claim 52, which has an altered IgG-antigenicity as compared to the parent Der f 2 polypeptide.

70. The variant of claim 52, which induces an altered immunogenic response in exposed animals as compared to the parent Der f 2 polypeptide.

71. The variant of claim 52, wherein the one or more mutations are independently substitutions of a hydrophilic amino acid to a hydrophobic amino acid, a polar amino acid to a non-polar amino acid, or an acidic amino acid to a basic amino acid.

72. The variant of claim 52, wherein the one or more mutations are insertions of one or more attachment groups for conjugating a polymer.

73. The variant of claim 52, wherein the one or more mutations are insertions of one or more glycosylation sites.

74. The variant of claim 52, which has at least the same T-cell stimulatory effect compared to the parent Der f 2 polypeptide.

75. A pharmaceutical composition comprising a variant of claim 52 and a pharmaceutically acceptable carrier or an adjuvant.

76. A nucleotide sequence encoding the variant of claim 52.

77. A nucleotide construct comprising the nucleotide sequence of claim 76, operably linked to one or more control sequences that direct the production of the variant in a host cell.

78. A recombinant expression vector comprising the nucleotide construct of claim 77.

79. A recombinant host cell comprising the nucleotide construct of claim 77.

80. A method of preparing a variant, comprising:
(a) cultivating the recombinant host cell of claim 79 under conditions conducive for production of the variant, and
(b) recovering the variant.


SEQUENCE LISTING (3 sequences)

SEQ ID NO: 1 - protein, 129 aa, Dermatophagoides pteronyssinus
  mat_peptide (1)..(129)
DQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 2 - protein, 129 aa, Dermatophagoides farinae
  mat_peptide (1)..(129)
DQVDVKDCANNEIKKVMVDGCHGSDPCIIHRGKPFTLEALFDANQNTKTAKIEIKASLDGLEIDVPGIDTNACHFMKCPLVKGQQYDIKYTWNVPKIAPKSENVVVTVKLIGDNGVLACAIATHGKIRD

SEQ ID NO: 3 - protein, 129 aa, Euroglyphus maynei
  PEPTIDE (1)..(129)
DQVDIKDCANHEIKKVMVPGCKGSEPCVIHRGTAFQLEAVFDANQNSNAAKIEIKATIDGVEIDVPGIDNNLCHFMKCPLVKGQEYDIKYTWNVPRIAPKSENVVVTVKLLGDNGVLACAIATHAKIRD